(19) United States
(12) Patent Application Publication
(10) Pub. No.: US 2022/0080001 A1
(43) Pub. Date: Mar. 17, 2022

(54) METHODS AND COMPOSITIONS FOR TREATING IMMUNE CHECKPOINT INHIBITOR ASSOCIATED COLITIS

(71) Applicant: Board of Regents, The University of Texas System, Austin, TX (US)

(72) Inventors: Jennifer A. Wargo, Houston, TX (US); Yinghong WANG, Houston, TX (US); Robert Jenq, Houston, TX (US)

(21) Appl. No.: 17/422,719

(22) PCT Filed: Jan. 16, 2020
(86) PCT No.: PCT/US20/13808

§ 371 Date: Jul. 13, 2021

Related U.S. Application Data
(60) Provisional application No. 62/793,085, filed on Jan. 16, 2019.

(51) Int. Cl. A61K 35/741; A61K 35/37

(52) U.S. Cl. CPC A61K 35/741; A61K 35/37

(57) ABSTRACT

Described herein are methods and compositions for treating immune checkpoint inhibitor (ICI)-associated colitis in a subject comprising administering fecal matter from a healthy donor to the subject. Further aspects of the disclosure relate to a method of treating immune checkpoint inhibitor (ICI)-associated colitis in a subject comprising administering to the subject a composition comprising at least one isolated or purified population of bacteria belonging to one or more of the genera Escherichia, Akkermansia, Bacteroides, Lachnospiraceae, Blautia, Tyzzerella, Bifidobacterium, Streptococcus, Colinsella, and Fusicatenibacter.

### CROSS-REFERENCE TO RELATED APPLICATIONS

This application claims the benefit of priority of U.S. Provisional Patent Application No. 62/793,085 filed Jan. 16, 2019, which is hereby incorporated by reference in its entirety.

### STATEMENT OF GOVERNMENT SUPPORT

This invention was made with government support under grant numbers CA219896 and HL124112 awarded by The National Institutes of Health. The government has certain rights in the invention.

### BACKGROUND OF THE INVENTION

### 1. Field of the Invention

This invention relates to the field of molecular biology and medicine.

### 2. Background

Immunotherapy has transformed the field of oncology, improving long-term survival in patients across numerous cancer types. Treatments with ICI targeting cytotoxic T-lymphocyte-associated antigen 4 (CTLA-4), programmed cell death protein 1 (PD-1), and programmed cell death ligand 1 (PD-L1) are associated with increased T-cell activation and effective antitumor immune responses in a subset of patients, but treatment can be associated with serious immune-related adverse effects (irAEs) in some patients. One of the most common toxicities is ICI-associated colitis. This can be quite severe and closely resembles colitis associated with autoimmune pathophysiology including inflammatory bowel disease (IBD). ICI-associated colitis is routinely treated with immunosuppressive therapy, including corticosteroids and/or agents targeting tumor-necrosis factor-α (TNF-α), all of which have significant side effects. Recommendations regarding optimal management of ICI-induced colitis continue to evolve. There is a need in the art for more effective treatments and/or for treatments with reduced side effects for ICI-associated colitis.

### SUMMARY OF THE INVENTION

Described herein are methods and compositions for treating immune checkpoint inhibitor (ICI)-associated colitis in a subject comprising administering fecal matter from a healthy donor to the subject.

Further aspects of the disclosure relate to a method of treating immune checkpoint inhibitor (ICI)-associated colitis in a subject comprising administering to the subject a composition comprising at least one isolated or purified population of bacteria belonging to one or more of the genera Escherichia, Akkermansia, Bacteroides, Lachnospiraceae, Blautia, Tyzzerella, Bifidobacterium, Streptococcus, Colinsella, and Fusicatenibacter.

In some embodiments, the composition comprises at least one isolated or purified population of bacteria belonging to one or more of the genera Akkermansia, Blautia, Bifidobacterium, Bacteroides, and Escherichia. In some embodiments, Escherichia comprises Escherichia shigella.

In some embodiments, the ICI-associated colitis comprises refractory ICI-associated colitis. In some embodiments, the subject has been treated with anti-CTLA-4 monotherapy. In some embodiments, the subject has been treated with anti-PD-1 monotherapy. In some embodiments, the subject has been treated with anti-CTLA-4 and anti-PD-1 combination therapy. In some embodiments, the colitis is classified as a Grade 2 or greater. In some embodiments, the subject has received a previous treatment for the ICI-associated colitis. In some embodiments, the subject has been determined to be unresponsive to the previous treatment. In some embodiments, the previous treatment comprises one or more of steroids, corticosteroids, anti-TNF-alpha therapy, anti-integrin therapy, infliximab, mesalamine, and vedolizumab. In some embodiments, the steroid comprises methylprednisolone or prednisolone.

In some embodiments, the subject has been determined to be unresponsive to intravenous methylprednisolone 140 mg/day for at least 5 days. In some embodiments, the subject has been determined to be unresponsive to intravenous methylprednisolone at at least or at most 50, 75, 100, 110, 120, 130, 140, 150, 160, 170, 180, 190, 200, 210, 220, 230, 240, 250, 260, 270, 280, 290, 300, 325, 350, 375, or 400 mg/day (or any derivable range therin) for at least or at most 1, 2, 3, 4, 5, 6, 7, 8, 9, 10, 11, 12, 13, 14, 15, 16, 17, 18, 19, 20, or 21 days (or any derivable range therein).

In some embodiments, the subject has been determined to be unresponsive or further unresponsive to at least one dose of 5 mg/kg of infliximab. In some embodiments, the subject has been determined to be unresponsive or further unresponsive to at least 1, 2, 3, 4, 5, or 6 doses (or any derivable range therein) of infliximab at at least 1, 2, 3, 4, 5, 6, 7, 8, 9, 10, 11, 12, 13, 14, 15, 16, 17, 18, 19, 20, 22, 24, 26, 28, 30, 32, 34, 36, 38, or 40 mg/kg per dose (or any derivable range therein). In some embodiments, the subject has been determined to be unresponsive or further unresponsive to a dose of 10 mg/kg infliximab.

In some embodiments, the subject has been determined to be unresponsive or further unresponsive to intravenous methylprednisolone 110 mg/day for at least 2 days. In some embodiments, the subject has been determined to be unresponsive or further unresponsive to intravenous methylprednisolone at at least 25, 50, 60, 70, 80, 90, 100, 110, 120, 130, or 140 mg/day (or any derivable range therein) for at least 1, 2, 3, 4, 5, 6, 7, 8, 9, 10, 11, 12, 13, 14, 15, 16, 17, 18, 19, 20, or 21 days (or any derivable range therein).

In some embodiments, the method comprises the administration of at least 2 doses of fecal matter. In some embodiments, the method comprises the administration of at least 2, 3, 4, 5, 6, 7, or 8 doses of fecal matter (or any derivable range therein). In some embodiments, the two administrations are at least 30 days apart. In some embodiments, the two administrations are at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 days (or any derivable range therein).

In some embodiments, the administration comprises intracolonic administration. In some embodiments, the administration comprises intracolonic administration to the cecum. In some embodiments, the method further comprises administration of one or more treatments. In some embodiments, the one or more treatments comprises one or more of corticosteroids, anti-TNF-alpha therapy, anti-integrin therapy, infliximab, mesalamine, and vedolizumab. In some embodiments, the method excludes one or more additional treatments after, at the most, 30 days post fecal matter administration. In some embodiments, the method excludes one or more additional treatments after, at the most, 10, 15, 20, 25, 30, 35, 40, 45, 50, 55, 60, 65 70, 75, 80, 85, 90, 95, or 100 days (or any derivable range therein) post fecal matter administration.

In some embodiments, the method excludes administration of steroids after 30 days post fecal matter administration. In some embodiments, the method excludes administration of steroids after at the most, 10, 15, 20, 25, 30, 35, 40, 45, 50, 55, 60, 65 70, 75, 80, 85, 90, 95, or 100 days (or any derivable range therein) post fecal matter administration.

In some embodiments, the healthy donor does not have cancer or has not been previously treated for cancer. In some embodiments, the healthy donor does not have colitis. In some embodiments, the subject has been diagnosed with refractory cancer. In some embodiments, the subject was administered immune checkpoint inhibitor therapy prior to administration of the fecal matter. In some embodiments, the subject is currently undergoing an immune checkpoint inhibitor therapy regimen. In some embodiments, the subject is administered immune checkpoint inhibitor therapy after administration of the fecal matter. In some embodiments, the administration of the immune checkpoint therapy and the fecal matter occurs within 7 days. In some embodiments, the administration of the immune checkpoint therapy and the fecal matter occurs within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days (or any derivable range therein).

In some embodiments, the fecal matter is administered in a dose of 50 g. In some embodiments, the fecal matter is administered in a dose of at least, at most, or exactly 10, 15, 20, 25, 30, 35, 40, 45, 50, 55, 60, 65, 70, 75. 80, 85, 90, 95, 100, 110, 120, 130, 140, 150, 160, 170, 180, 190, 200, 210, 220, 230, 240, 250, 260, 270, 280, 290, 300, 325, 350, 375, or 400 g (or any derivable range therein).

In another aspect, the disclosure relates to compositions comprising an isolated or purified population of at least one, at least two, or 3, 4, 5, 6, 7, 8, 9, 10, 15, or 20 (or any derivable ragnge therein) of Bacteroides stercoris, Bacteroides caccae, Bacteroides intestinalis, Dialister, Bacteroides fragilis, Vampirovibrio, Tyzzerella, Bacteroides stercoris, Flavonifractor plautii, Dielma fastidiosa, Akkermansia muciniphila, Lactobacillus rogosae, Bacteroides fragilis, Prevotella copri, Prevotella shahii Firmicutes, Clostridiales, Ruminococcaceae, Alistipes indistinctus, Bacteroides stercorirosoris, Clostridium lactatifermentans orus, Abyssivirga alkaniphila, Acetatifactor muris, Acetivibrio cellulolyticus, Acetivibrio ethanolgignens, Acholeplasma vituli, Achromobacter deleyi, Acidovorax radices, Adlercreutzia equolifaciens, Akkermansia muciniphila, Alistipes indistinctus, Alistipes obesi, Alistipes putredinis, Alistipes senegalensis, Alistipes timonensis, Alkalibacter saccharofermentans, Alkalibaculum bacchi, Allobaculum stercoricanis, Anaerobacterium chartisolvens, Anaerocolumna cellulosilytica, Anaerosporobacter mobilis, Anaerotaenia torta, Anaerotruncus colihominis, Anaerotruncus rubiinfantis, Anaerovorax odorimutans, Bacteroides acidifaciens, Bacteroides caecimuris, Bacteroides dorei, Bacteroides faecichinchillae, Bacteroides rodentium, Bacteroides stercorirosoris, Bacteroides xylanolyticus, Barnesiella intestinihominis, Beduini massiliensis, Bifidobacterium pseudolongum, Blautia luti, Breznakia blatticola, Breznakia pachnodae, Butyricicoccus pullicaecorum, Butyrivibrio crossotus, Catabacter hongkongensis, Christensenella massiliensis, Christensenella minuta, Christensenella timonensis, Clostridium aerotolerans, Clostridium aldenense, Clostridium alkalicellulosi, Clostridium asparagiforme, Clostridium celerecrescens, Clostridium cellobioparum, Clostridium cellulolyticum, Clostridium clariflavum, Clostridium cocleatum, Clostridium colinum, Clostridium hylemonae, Clostridium indolis, Clostridium jejuense, Clostridium lactatifermentans, Clostridium lavalense, Clostridium methylpentosum, Clostridium oroticum, Clostridium oryzae, Clostridium papyrosolvens, Clostridium polysaccharolyticum, Clostridium populeti, Clostridium saccharolyticum, Clostridium saudiense, Clostridium scindens, Clostridium straminisolvens, Clostridium viride, Clostridium xylanolyticum, Coprobacter secundus, Coprococcus catus, Culturomica massiliensis, Defluviitalea saccharophila, Desulfitobacterium hafniense, Desulfitobacterium metallireducens, Desulfosporosinus orientis, Desulfovibrio desulfuricans, Desulfovibrio simplex, Dorea formicigenerans, Eisenbergiella massiliensis, Emergencia timonensis, Enterococcus hirae, Enterorhabdus mucosicola, Enterorhabdus muris, Erysipelatoclostridium ramosum, Erysipelothrix larvae, Escherichia fergusonii, Eubacterium coprostanoligenes, Eubacterium dolichum, Eubacterium ruminantium, Eubacterium siraeum, Eubacterium tortuosum, Eubacterium ventriosum, Faecalibaculum rodentium, Flavimarina pacifica, Flavonifractor plautii, Flintibacter butyricus, Gordonibacter faecihominis, Gracilibacter thermotolerans, Harryflintia acetispora, Holdemania massiliensis, Hydrogenoanaerobacterium saccharovorans, Ihubacter massiliensis, Intestinimonas butyriciproducens, Irregularib acter muris, Lachnoclostridium pacaense, Lactobacillus animalis, Lactobacillus faecis, Lactobacillus gasseri, Lactobacillus hominis, Lactobacillus intestinalis, Lactobacillus johnsonii, Lactobacillus reuteri, Lactobacillus rogosae, Lactobacillus taiwanensis, Lawsonia intracellularis, Longibaculum muris, Marvinbryantia formatexigens, Millionella massiliensis, Mucispirillum schaedleri, Muribaculum intestinale, Murimonas intestini, Natranaerovirga pectinivora, Neglecta timonensis, Odoribacter splanchnicus, Olsenella profusa, Oscillibacter ruminantium, Oscillibacter valericigenes, Papillibacter cinnamivorans, Parabacteroides goldsteinii, Paraeggerthella hongkongensis, Parasutterella excrementihominis, Parvibacter caecicola, Peptococcus niger, Phocea massiliensis, Porphyromonas catoniae, Prevotella oralis, Prevotella stercorea, Prevotellamassilia timonensis, Pseudobutyrivibrio ruminis, Pseudoflavonifractor capillosus, Pseudoflavonifractor phocaeensis, Raoultibacter timonensis, Rhizobium straminoryzae, Roseburia faecis, Roseburia hominis, Roseburia intestinalis, Ruminiclostridium thermocellum, Ruminococcus champanellensis, Ruminococcus faecis, Ruminococcus flavefaciens, Ruminococcus gnavus, Ruthenibacterium lactatiformans, Sphingomonas kyeonggiensis, Spiroplasma velocicrescens, Sporobacter termitidis, Stomatobaculum longum, Streptococcus acidominimus, Streptococcus danieliae, Syntrophomonas wolfei, Tepidimonas taiwanensis, Tindallia californiensis, Tindallia texcoconensis, Turicibacter sanguinis, Turicimonas muris, Tyzzerella nexilis, Vallitalea pronyensis, and/or Vampirovibrio chlorellavorus.

In some embodiments, the cancer is a skin cancer. In some embodiments, the cancer is basal-cell skin cancer, squamous-cell skin cancer, melanoma, dermatofibrosarcoma protuberans, Merkel cell carcinoma, Kaposi's sarcoma, keratoacanthoma, spindle cell tumors, sebaceous carcinomas, microcystic adnexal carcinoma, Paget' s disease of the breast, atypical fibroxanthoma, leiomyosarcoma, or angiosarcoma. In some embodiments, the cancer is melanoma. In some embodiments, the melanoma is metastatic melanoma, Lentigo Maligna, Lentigo Maligna Melanoma, Superficial Spreading Melanoma, Nodular Melanoma, Acral Lentiginous Melanoma, Cutaneous Melanoma, or Desmoplastic Melanoma. In some embodiments, the cancer comprises Cutaneous Melanoma.

In some embodiments, the cancer comprises recurrent cancer. In some embodiments, the cancer comprises recurrent metastatic cancer. In some embodiments, the cancer comprises a recurrence of the cancer in the area of the primary tumor. In some embodiments, the cancer comprises a metastatic cancer. In some embodiments, the cancer comprises a stage III or IV cancer. In some embodiments, the cancer comprises a stage I or II cancer. In some embodiments, the cancer excludes stage I or II cancer.

In some embodiments, the method further comprises administering at least one additional anticancer treatment. In some embodiments, the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy. In some embodiments, the additional anticancer treatment comprises a cancer treatment described herein.

In some embodiments, the therapy, microbial composition, or fecal matter is administered intratumorally, intraarterially, intravenously, intravascularly, intrapleurally, intraperitoneally, intratracheally, intrathecally, intramuscularly, endoscopically, intralesionally, percutaneously, subcutaneously, regionally, stereotactically, orally or by direct injection or perfusion. In some embodiments, the route of administration is a route described herein.

In some embodiments, administration of the fecal matter or microbial composition provides for a reduction in CD8+ T-cell density or in CD8+ cytotoxic T lymphocytes. In some embodiments, administration of the fecal matter or microbial composition provides for a reduction of at least 2, 3 4, 5, 6, 7, 8, 9, 10, 15, 20, 25, 30, 35, 40, 45, 50, 55, 60, 65, or 70% (or any derivable range therein) in CD8+ T-cell density or in CD8+ cytotoxic T lymphocytes. In some embodiments, administration of the fecal matter or microbial composition provides for an increase in CD4+ FoxP3+. In some embodiments, administration of the fecal matter or microbial composition provides for an increase of at least 2, 3 4, 5, 6, 7, 8, 9, 10, 15, 20, 25, 30, 35, 40, 45, 50, 55, 60, 65, or 70% (or any derivable range therein) in CD4+ FoxP3+.

In some embodiments, the purified population of bacteria comprises bacteria from at least two genera or species, and wherein the ratio of the two bacteria is 1:1. In some embodiments, the purified population of bacteria comprises bacteria from at least, at most, or exactly 1, 2, 3, 4, 5, 6, 7, 8, 9, 10, 11, 12, 14, 16, 20, 30, 40, or 50 (or any derivable range therein) different families, genera, or species of bacteria. In some embodiments, the ratio of one family, genera, or species of bacteria to another family, genera, or species of bacteria present in the composition is at least, at most, or exactly 1:1, 1:2, 1:3, 1:4, 1:5, 1:6, 1:7, 1:8, 1:9, 1:10, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:600, 1:700, 1:800, 1:900, 1:1000, 1:1500, 1:2000, 1:2500, 1:3000, 1:3500, 1:4000, 1:4500, 1:5000, 1:1550, 1:6000, 1:6500, 1:7000, 1:7500, 1:8000, 1:8500, 1:9000, 1:9500, 1:10000, 1:1200, 1:14000, 1:16000, 1:18000, 1:20000, 1:30000, 1:40000, 1:50000, 1:60000, 1:70000, 1:80000, 1:90000, or 1:100000 (or any derivable range therein).

In some embodiments, the compositions provide for an alpha diversity of at least, at most, or exactly 1, 2, 3, 4, 5, 6, 7, 8, 9, 10, 11, 12, 13, 14, 15, 16, 17, 18, 19, or 20. Methods of calculating alpha diversity are known in the art. For example, taxonomic alpha-diversity of samples can be estimated using the Inverse Simpson Index. In some embodiments, the compositions are administered in an effective amount. In some embodiments, the effective amount comprises an amount that provides for an an alpha diversity of at least, at most, or exactly 1, 2, 3, 4, 5, 6, 7, 8, 9, 10, 11, 12, 13, 14, 15, 16, 17, 18, 19, or 20 (or any derivable range therein) in the subject.

In some embodiments, the bacteria belonging to the genera or species Escherichia, Akkermansia, Bacteroides, Lachnospiraceae, Blautia, Tyzzerella, Bifidobacterium, Streptococcus, Colinsella, and/or Fusicatenibacter are administered in an amount of at least, at most, or exactly 1×10³, 1×10⁴, 1×10⁵, 1×10⁶, 1×10⁷, 1×10⁸, 1×10⁹, 1×10¹⁰ , 1×10¹¹, 1×10¹², 1×10¹³, 1×10¹⁴, 1×10¹⁵, or 1×10¹⁶ cells or CFU (or any derivable range therein). In some embodiments, the total amount of bacteria administered is at least, at most, or exactly 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ cells or CFU (or any derivable range therein). In some embodiments, a particular amount of bacteria such as a particular species of bacteria may be at least, at most, or exactly in an amount of at least, at most, or exactly 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ cells or CFU (or any derivable range therein). In some embodiments, the composition may contain at least, at most, or exactly at least, at most, or exactly 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ cells or CFU (or any derivable range therein)from a phylum, family, genera or species of bacteria described herein. In some embodiments, the composition may contain less than at least, at most, or exactly 1×10⁶, 1×10⁵, 1×10⁴, 1×10³, or 1×10² cells or CFU (or any derivable range therein) from a phylum, family, genus or species of bacteria described herein.

In some embodiments, the method further comprises administration of an antibiotic. In some embodiments, the antibiotic may be a broad spectrum antibiotic. In some embodments, a mixture of at least 1, 2, 3, 4, or 5 antibiotics is administered. In some embodiments, the antibiotics comprises ampicillin, streptomycin, and colistin, and combinations thereof. In some embodiments, the antibiotic is administered prior to the composition comprising at least one isolated or purified population of bacteria. In some embodiments, the antibiotic is administered concurrent with the composition comprising at least one isolated or purified population of bacteria. In some embodiments, the antibiotic is administered at least or at most 1, 2, 3, 4, 5, 6, 7, 8, 9, 10, 12, or 24 hours or 1 2, 3, 4, 5, or 6 days or 1, 2, 3, 4, 5, or 6 weeks (or any derivable range therein) before or after the microbial composition.

The compositions of the disclosure may exclude one or more bacteria genera or species described herein or may include less than 1×10⁶, 1×10⁵, 1×10⁴, 1×10³, or 1×10² cells or CFU (or any derivable range therein) of one or more of the bacteria described herein.

In some embodiments, each of the populations of bacteria is present in the composition at a concentration of at least 10\hat{ }3 CFU. In some embodiments, the composition is a live bacterial product or a live biotherapeutic product. In some embodiments, the compositions of the disclosure, such as a composition comprising a microbial populations or fecal matter are lyophilized, freeze dried, or frozen. In some embodiments, the composition is formulated for oral delivery. In some embodiments, the composition formulated for oral delivery is a tablet or capsule. In some embodiments, the tablet or capsule comprises an acid-resistant enteric coating. In some embodiments, the composition is formulated for administration rectally, via colonoscopy, sigmoidoscopy by nasogastric tube, or enema. In some embodiments, the composition is capable of being reformulated for final delivery as comprising a liquid, a suspension, a gel, a geltab, a semisolid, a tablet, a sachet, a lozenge, a capsule, or as an enteral formulation. In some embodiments, the composition is formulated for multiple administrations. In some embodiments, the composition further comprises a pharmaceutically acceptable excipient.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Is is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in to context of the term “consisting of” or “consisting essentially of”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-F. Endoscopic changes and characterization of colonic mucosal infiltrate throughout clinical course. Patient 1. a, Changes in colonic mucosa as assessed by full colonoscopy. Near the time of diagnosis (row 1), multiple large ulcers and diffuse inflammatory exudate are present (in the distal 40 cm of the colon only, with normal appearing proximal colon) and remain despite months of treatment with steroids and biologic immunosuppressive agents (steroids+two doses infliximab+one dose vedolizumab) (row 2). Approximately 1 month after FMT (row 3), the colonic mucosa exhibits grossly normal vasculature, minimal patchy erythema, and near-complete healing of prior ulcers. Yellow arrows point to ulcerative lesions. This patient had full colonoscopic evaluations that examined every segment of the colon (ascending, transverse, descending, and sigmoid colon, and rectum). Endoscopy was performed once at each time point. Given the qualitative nature of endoscopic data collection and inability to provide true statistical analyses, the inventors chose to include multiple other representative photos from the same colonoscopic evaluations (FIG. 6A). b, IHC analysis of mucosal biopsies of the colon/rectum prior to, and following, FMT. A single slide representative of the endoscopic biopsy specimen as a whole was stained for each patient for each time point. Representative slides from additional time points are included in FIGS. 7A and 7C, Analysis of changes in the density immune cell subsets (CD8 red squares, CD4 blue circles, FOXP3 black triangles) over time, expressed as a fold change from baseline based on total densities of cells expressing these markers (absolute densities are presented in FIG. 8A). Time points include time of diagnosis, priorto FMT, following steroids and biologic immunosuppression and following FMT. Date of FMT is represented by dotted vertical line and is designated day 0. These data represent the average cell density from four ROIs per sample (single slide per patient at each time point) with each ROI measuring 500×500 μm² for a total of approximately 1 mm². The inventors report the mean number of IHC-positive cells per mm² divided by the mean number of IHC-positive cells per mm² at baseline. Patient 2. d, Changes in colonic mucosa as assessed by full colonoscopy. Near the time of diagnosis (row 1), multiple large ulcers and inflammatory exudate are present (throughout the entire colon) and remain after unsuccessful treatment with steroids and biologic immunosuppressive agents (steroid+two doses infliximab+four doses vedolizumab) (row 2). There is notable improvement following first FMT (row 3) but residual ulcers remain. Following second FMT (row 4), the inventors note near-complete resolution of all ulcerative lesions. Again full endoscopic examinations were performed, once for each time point. Additional representative photographs from the colonoscopic evaluation are shown in FIG. 6B. e, Immunohistochemical analysis of mucosal biopsies of the colon/rectum taken prior to first FMT and following first FMT. A single slide representative of the endoscopic biopsy specimen was stained for each patient for each time point. Representative slides from additional time points are included in FIGS. 7B and 7F, Analysis of changes in the density immune cell subsets (CD8 red squares, CD4 blue circles, FOXP3 black triangles) over time, expressed as a fold change from baseline based on total densities of cells expressing these markers (absolute densities are presented in FIG. 8B). These data represent the average cell density from four ROIs per sample (single slide per patient at each time point) with each ROI measuring 500×500 μm² for a total of approximately 1mm². The inventors report the mean number of IHC-positive cells per mm² divided by the mean number of IHC-positive cells per mm² at baseline. Date of first (day 0) and second FMT (day 67) are represented by dotted vertical lines.

FIG. 2A-E. Microbiome analysis of patient and donor intestinal bacteria by 16S deep sequencing. The patients' stool microbiomes were longitudinally sampled at indicated time points before and after FMT, along with samples from the FMT donor. Between 3,380 and 42,776 sequences were obtained for each sample (average 10,003). a, α-diversity, quantified by the inverse Simpson's index after rarefying to 3,000 sequences, as well as total observed OTU numbers, was evaluated for patient and FMT donor samples. b, Using principal coordinate analysis (PCoA) of unweighted UniFrac distances, microbiome samples from a are depicted in space with more similar samples located closer together. c, Bacterial 16S sequences from samples in a were classified by origin (unique to patient baseline, unique to donor, present in both patient baseline and donor, or absent in both patient baseline and donor). d, Sequences were classified by taxonomy at the Class level. e, Changes in the abundances of the top ten varying ten bacterial genera over time.

FIG. 3. Timeline representing the clinical course of Patient 1. Key timepoints include timing of immunotherapy, diagnosis of colitis, length of time treated with traditional agents including steroids (initially dosed at 2 mg/kg IV and subsequently weaned slowly over the course of months), and other immunosuppressive agent including infliximab and vedolizumab, time of FMT. We also denote times during which endoscopy was performed and images are shown (**). Biopsies were taken for immunohistochemical analysis at designated times (##). We also denote timepoints at which time fecal material was collected for analysis of the gut microbiome. Below the timeline, are the approximate dates and duration of different antibiotic therapies the patients received for various clinical indications throughout this time course.

FIG. 4. Timeline representing the clinical course of Patient 2. Key timepoints include timing of immunotherapy, diagnosis of colitis, length of time treated with traditional agents including steroids (initially dosed at 2 mg/kg IV and subsequently weaned slowly over the course of months), and other immunosuppressive agent including infliximab and vedolizumab, and timing of first and second FMT. We establish the time of first FMT as Day 0 and report other dates relative to this key time point. We denote times during which endoscopy was performed and images shown (**). Biopsies were taken for immunohistochemical analysis at times marked by ##. We also denote timepoints at which time fecal material was collected for analysis of the gut microbiome. Below the timeline, are the approximate dates and duration of different antibiotic therapies the patients received for various clinical indications throughout this time course. Additionally, we denote timing of other chemotherapeutic agents administered during this time period.

FIG. 5A-B. Severity of colitis. Various measures of disease severity are plotted throughout clinical course for (a) Patient 1 and (b) Patient 2, including daily dosage of systemic steroids (blue line), grade of diarrhea (red squares) and colitis (black triangles) as assessed by CTCAE Version 4 and endoscopic severity score (light blue diamonds) which incorporates presence of erythema/erosions, presence of ulcer, and number (≥2), size (≥1 cm) and depth (≥2 mm) of mucosal ulcerations (each feature counts one point). Vertical dotted line indicates date of FMT. The endoscopic scoring criteria was created based on institutional expertise. Timing of doses of immunosuppressive agents is also noted.

FIG. 6A-B. Additional endoscopic images from colonoscopy. (a) For Patient 1, images of colon and rectum near the time of diagnosis (row 1), after unsuccessful treatment with steroids and biologic immunosuppressive agents (steroid+2 doses infliximab+1 dose vedolizumab) (row 2) and approximately one month after FMT (row 3). The ulcers and inflammation were diffusely distributed in the distal 40 cm of the colon. The rest of the proximal colon appeared normal based on full colon endoscopy exam. (b) For Patient 2, images of colon and rectum near the time of diagnosis (row 1), after unsuccessful treatment with steroids and biologic immunosuppressive agents (steroid+2 doses infliximab+4 doses vedolizumab) (row 2), approximately 5 weeks following the first FMT (row 3), and approximately three months after the second FMT (row 4). Yellow arrows point to ulcerative lesions. The ulcers and inflammation were throughout the entire colon with patchy distribution pattern. Each photo represents a unique portion of the colon and rectum. It is important to note that the photos directly beneath one another in subsequent rows do not necessarily correlate to the exact same location within the colon or rectum. A single endoscopic exam was performed at each time point.

FIG. 7A-B. Immunohistochemical analysis of colonic mucosa. (a) For Patient 1, representative slides from multiple endoscopic biopsies taken at each of clinically relevant time points including at time of diagnosis, following unsuccessful treatment with steroids and 2 doses of infliximab, following additional one dose of vedolizumab, and following FMT (b) For Patient 2, representative slides from multiple biopsies taken at diagnosis, following unsuccessful treatment with steroids and biologic immunosuppression, and following first FMT and following second FMT. Shown is H&E staining for each as well as staining for individual markers common to T lymphocytes: CD8, CD4 and FoxP3. Date of FMT1 is considered as Day 0. For both patients, a single representative slide of the endoscopic biopsy specimen as a whole was stained per time point. For each sample, we analyzed 4 regions of interest (ROI). Each ROI measured 0.5 mm×0.5 mm (for a total of ˜1.00 mm2).

FIG. 8A-B. Quantification of immunohistochemical analysis of colonic mucosa (a) For Patient 1, absolute densities of different immune cells (cells/mm2) at time of diagnosis, during initial treatment with steroids and 2 doses of infliximab, following additional one dose of vedolizumab treatment, and following FMT. Vertical dotted line represents timing of the FMT (Day 0). (b) For Patient 2, absolute densities of different immune cells (cells/mm2) at time of diagnosis, following unsuccessful treatment with steroids and biologic immunosuppression, following FMT 1 and following the second FMT. For both patients, a single slide representative of the endoscopic biopsy specimen as a whole was stained per time point. These data represent the average (+/−standard deviation) cell density from 4 regions of interest per sample with each ROI measuring 500 μm×500 μm. Vertical dotted line represents timing of FMT for each patient. Date of FMT1 is considered as Day 0.

FIG. 9. Colocalization of CD4 and FoxP3. Representative multiplex IHC demonstrating distinct CD8+ (red) and CD4+ (yellow) lymphocyte populations as well as co-localization of CD4+ and FoxP3 (green) in multiple cells which likely represent T regulatory lymphocytes. DAPI staining of nuclei in blue. This represents biopsy from Patient 2 following FMT1. Multiple replicates (2 or more) of at least two time points for each patient demonstrated similar results.

### DESCRIPTION OF ILLUSTRATIVE EMBODIMENTS

### I. DEFINITIONS

As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. Antibodies used with the methods and compositions described herein are generally derivatives of the IgG class. The term antibody also refers to antigen-binding antibody fragments. Examples of such antibody fragments include, but are not limited to, Fab, Fab′, F(ab′)2, scFv, Fv, dsFv diabody, and Fd fragments. Antibody fragments may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment retains the ability to bind its cognate antigen at comparable affinity to the full antibody.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include several different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical composition” or “pharmacologically acceptable composition” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition may be adjusted according to well-known parameters.

The term “unit dose” or “dosage” refers to physically discrete units suitable for use in a subject, each unit containing a predetermined quantity of the therapeutic composition calculated to produce the desired responses discussed herein in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any particular dose derivable therein. In non-limiting examples of a range derivable from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

A “population” of bacteria may refer to a composition of bacteria comprising a single species, or a mixture of different species?

The term “immune checkpoint” refers to a component of the immune system which provides inhibitory signals to its components in order to regulate immune reactions. Known immune checkpoint proteins comprise CTLA-4, PD-1 and its ligands PD-L1 and PD-L2 and in addition LAG-3, BTLA, B7H3, B7H4, TIM3, KIR. The pathways involving LAG3, BTLA, B7H3, B7H4, TIM3, and KIR are recognized in the art to constitute immune checkpoint pathways similar to the CTLA-4 and PD-1 dependent pathways (see e.g. Pardoll, 2012, Nature Rev Cancer 12:252-264; Mellman et al., 2011, Nature 480:480-489).

The term “inhibitor” refers to a molecule that may be organic or inorganic, a protein, polypeptide, antibody, small molecule, carbohydrate, or nucleic acid that blocks or decreases one or more functions of the protein. The inhibitor may be a direct inhibitor that acts by directly interacting with the protein or an indirect inhibitor that may not interact directly with the protein but still inhibits one or more functions of the protein.

An “immune checkpoint inhibitor” refers to any compound inhibiting the function of an immune checkpoint protein. Inhibition includes reduction of function and full blockade.

In particular the immune checkpoint protein is a human immune checkpoint protein. Thus the immune checkpoint protein inhibitor in particular is an inhibitor of a human immune checkpoint protein.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

The “gut microbiota” or “gut microbiome” designates the population of microorganisms (and their genomes) living in the intestine of a subject.

The term “alpha diversity” is a measure of intra-sample diversity and refers to the distribution and assembly patterns of all microbiota within samples and is calculated as a scalar value for each sample. “Beta diversity” is a term for inter-sample diversity, and involves the comparison of samples to each which provides a measure of the distance or dissimilarity between each sample pair.

The term “relative amount”, which can also be designated as the “relative abundance”, is defined as the number of bacteria of a particular taxonomic level (from phylum to species) as a percentage of the total number of bacteria of that level in a biological sample. This relative abundance can be assessed, for example, by measuring the percentage of 16S rRNA gene sequences present in the sample which are assigned to these bacteria. It can be measured by any appropriate technique known by the skilled artisan, such as 454 pyrosequencing of the specific bacterial 16S rRNA gene markers or quantitative PCR of a specific gene.

The term “isolated” encompasses a bacterium or other entity or substance that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The terms “purify,” “purifying” and “purified” refer to a bacterium or other material that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, and a purified bacterium or bacterial population may contain other materials up to about 10%, about 20%, about 30%, about 40%, about 50%, about 60%, about 70%, about 80%, about 90%, or above about 90% and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

The phrase “effective amount” or “therapeutically effective amount” or “sufficient amount” means a dosage of a drug or agent sufficient to produce a desired result. The desired result can be a decrease in tumor size, a decrease in the rate of growth of cancer cells, a decrease in metastasis, increase in CD8+ T lymphocytes in the tumor or tumor immune infiltrate, an increase in CD45+, CD3+/CD20+/CD56+, CD68+ and/or HLA-DR+ cells in the tumor, an increase in CD3, CD8, PD1, FoxP3, Granzyme B and/or PD-L1 expression in a tumor immune infiltrate, a decrease in RORγT expression in a tumor immune infiltrate, an increase of effector CD4+, CD8+ T, monocytes and/or myeloid dendritic cell in the systemic circulation or the peripheral blood, a decrease of B cells, regulatory T cells and/or myeloid derived suppressor cells in the systemic circulation or the peripheral blood of the subject or any combination of the above.

### II. CHECKPOINT INHIBITORS AND COMBINATION TREATMENT

In some embodiments of any one of the methods, compositions or kits provided, the immune checkpoint inhibitor is a small molecule inhibitor. In some embodiments of any one of the methods, compositions or kits provided, the immune checkpoint inhibitor is a polypeptide that inhibits an immune checkpoint pathway. In some embodiments of any one of the methods, compositions or kits provided, the inhibitor is a fusion protein. In some embodiments of any one of the methods, compositions or kits provided, the immune checkpoint inhibitor is an antibody. In some embodiments of any one of the methods, compositions or kits provided, the antibody is a monoclonal antibody.

A. PD-1, PDL1, and PDL2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the antibody described herein (such as an anti-PD-1 antibody, an anti-PDL1 antibody, or an anti-PDL2 antibody) further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further specific aspect, the antibody has reduced or minimal effector function. In a still further specific aspect, the minimal effector function results from production in prokaryotic cells. In a still further specific aspect the minimal effector function results from an “effectorless Fc mutation” or alpha-glycosylation.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Accordingly, an antibody used herein can comprise alpha-glycosylation. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxy amino acid, most commonly serine or threonine, although 5-hydroxyproline or 5 -hydroxy lysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

The antibody or antigen binding fragment thereof, may be made using methods known in the art, for example, by a process comprising culturing a host cell containing nucleic acid encoding any of the previously described anti-PDL1, anti-PD-1, or anti-PDL2 antibodies or antigen-binding fragment in a form suitable for expression, under conditions suitable to produce such antibody or fragment, and recovering the antibody or fragment.

B. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Patent No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Other molecules for modulating CTLA-4 include soluble CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

### III. MICROBIAL MODULATORS

Embodiments of the present disclosure concern microbial modulator compositions for the treatment of colitis and in particular in methods for modifying the microbiome of subjects that have been treated with or will be treated with combination immune checkpoint inhibitor therapy.

The present disclosure also provides a pharmaceutical composition comprising one or more microbial cultures as described above. The bacterial species therefore are present in the dose form as live bacteria, whether in dried, lyophilized, or sporulated form. This may be preferably adapted for suitable administration; for example, in tablet or powder form, potentially with an enteric coating, for oral treatment.

In particular aspects, the composition is formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

While the compositions of the present disclosure are preferably formulated for oral administration, other routes of administration can be employed, however, including, but not limited to, subcutaneous, intramuscular, intradermal, transdermal, intraocular, intraperitoneal, mucosal, vaginal, rectal, and intravenous.

The desired dose of the composition of the present disclosure may be presented in multiple (e.g., two, three, four, five, six, or more) sub-doses administered at appropriate intervals throughout the day.

In one aspect, the disclosed composition may be prepared as a capsule. The capsule (i.e., the carrier) may be a hollow, generally cylindrical capsule formed from various substances, such as gelatin, cellulose, carbohydrate or the like.

In another aspect, the disclosed composition may be prepared as a suppository. The suppository may include but is not limited to the bacteria and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In some aspects, the disclosed microbial modulator composition may be prepared as a tablet. The tablet may include the bacteria and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets.

In other aspects, the disclosed microbial modulator composition may be formed as food or drink or, alternatively, as an additive to food or drink, wherein an appropriate quantity of bacteria is added to the food or drink to render the food or drink the carrier.

The microbial modulator compositions of the present disclosure may further comprise one or more prebiotics known in the art, such as lactitol, inulin, or a combination thereof.

In some embodiments, the microbial modulator composition may further comprise a food or a nutritional supplement effective to stimulate the growth of bacteria of the order Clostridiales present in the gastrointestinal tract of the subject. In some embodiments, the nutritional supplement is produced by a bacterium associated with a healthy human gut microbiome.

### IV. ADDITIONAL THERAPIES

The current methods and compositions of the disclosure may include one or more additional therapies known in the art and/or described herein. In some embodiments, the additional therapy comprises an additional cancer treatment. Examples of such treatments are described herein.

A. Immunotherapies

In some embodiments, the additional therapy comprises a further cancer immunotherapy. Cancer immunotherapy (sometimes called immunooncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death. [60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable.

In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. ADMINISTRATION OF THERAPEUTIC COMPOSITIONS

Methods of the disclosure include administration of a combination of therapeutic agents and/or administration of therapeutic agents, such as fecal matter and therapeutic regimens, such as steroid therapy or anti-integrin therapy, for example. The therapy may be administered in any suitable manner known in the art. For example, the therapies may be administered sequentially (at different times) or concurrently (at the same time). In some embodiments, the therapies are in a separate composition. In some embodiments, the therapies are in the same composition.

Various combinations of the therapies may be employed, for example, one therapy designated “A” and another thapy designated “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapies of the disclosure, such as the fecal matter from a healthy subject may be administered by the same route of administration or by different routes of administration. In some embodiments, the therapy is administered intracolonically, intravenously, intramuscularly, subcutaneously, topically, orally, transdermally, intraperitoneally, intraorbitally, by implantation, by inhalation, intrathecally, intraventricularly, or intranasally. In some embodiments, the microbial modulator is administered intravenously, intramuscularly, subcutaneously, topically, orally, transdermally, intraperitoneally, intraorbitally, by implantation, by inhalation, intrathecally, intraventricularly, or intranasally.

For example, the therapeutically effective or sufficient amount of each of the at least one isolated or purified population of bacteria or each of the at least two, 3, 4, 5, 6, 7, 8, 9, 10 11, 12, 13, 14, or 15 isolated or purified populations of bacteria of the microbial modulator compositions of the embodiments that is administered to a human will be at least about 1×10e3 colony forming units (CFU) of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU (or any derivable range therein). In some embodiments, a single dose will contain an amount of bacteria (such as a specific bacteria or species, genus, or family described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ CFU (or any derivable range therein) of a specified bacteria. In some embodiments, a single dose will contain at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ CFU (or any derivable range therein) of total bacteria. In specific embodiments, the bacteria are provided in spore form or as sporulated bacteria. In particular embodiments, the concentration of spores of each isolated or purified population of bacteria, for example of each species, subspecies or strain, is at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ (or any derivable range therein) viable bacterial spores per gram of composition or per administered dose. In some embodiments, the composition comprises or the method comprises administration of at least, at most, or exactly 1, 2, 3, 4, 5, 6, 7, 8, 9, 10, 11, 12, 13, 14, 15, 20, 25, 30, 40, or 50 (or any derivable range therein) of different bacterial species, different bacterial genus, or different bacterial family.

In some embodiments, the therapeutically effective or sufficient amount of each of the at least one isolated or purified population of bacteria or each of the at least two, 3, 4, 5, 6, 7, 8, 9, 10 11, 12, 13, 14, or 15 isolated or purified populations of bacteria of the microbial modulator compositions of the embodiments that is administered to a human will be at least about 1×103 cells of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range therein). In some embodiments, a single dose will contain an amount of bacteria (such as a specific bacteria or species, genus, or family described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ cells (or any derivable range therein) of a specified bacteria. In some embodiments, a single dose will contain at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ cells (or any derivable range therein) of total bacteria. In specific embodiments, the bacteria are provided in spore form or as sporulated bacteria. In particular embodiments, the concentration of spores of each isolated or purified population of bacteria, for example of each species, subspecies or strain, is at least, at most, or exactly 1×10⁴, 1×10⁵, 1×10⁶, 1×10⁷, 1×10⁸, 1×10⁹, 1×10¹⁰ , 1×10¹¹, 1×10¹², 1×10¹³, 1×10¹⁴, 1×10¹⁵ or greater than 1×10¹⁵ (or any derivable range therein) viable bacterial spores per gram of composition or per administered dose. In some embodiments, the composition comprises or the method comprises administration of at least, at most, or exactly 1, 2, 3, 4, 5, 6, 7, 8, 9, 10, 11, 12, 13, 14, 15, 20, 25, 30, 40, or 50 (or any derivable range therein) of different bacterial species, different bacterial genus, or different bacterial family.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administerable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of a therapeutic composition that is administered to a human will be in the range of about 0.01 to about 50 mg/kg of patient body weight whether by one or more administrations. In some embodiments, the therapeutic agent used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the therapeutic agent is administered at 15 mg/kg. However, other dosage regimens may be useful. In one embodiment, a therapeutic agent described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VI. METHODS OF TREATMENT

Provided herein are methods for treating or delaying progression of colitis by administration of therapeutic compositions, such as compositions comprising fecal matter from a healthy subject, to a subject who has been or is currently being administered immune checkpoint therapy.

In some embodiments, the treatment results in a sustained response in the individual after cessation of the treatment. In some embodiments, the individual has cancer or has colitis that is resistant (has been demonstrated to be resistant) to one or more anti-cancer therapies. In some embodiments, resistance to therapy includes recurrence of or refractory colitis.

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some embodiments, the treatment may exclude prevention of the disease.

In certain aspects, further cancer or metastasis examination or screening, or further diagnosis such as contrast enhanced computed tomography (CT), positron emission tomography-CT (PET-CT), and magnetic resonance imaging (MRI) may be performed for the detection of cancer or cancer metastasis in patients determined to have a certain gut microbiome composition.

### VII. METHODS OF DETERMINING MICROBIOME COMPOSITION

In some embodiments, the methods relate to obtaining a microbiome profile. In some embodiments, obtaining a microbiome profile comprises the steps of or the ordered steps of: i) obtaining a sample obtained from a subject (e.g., a human subject), ii) isolating one or more bacterial species from the sample, iii) isolating one or more nucleic acids from at least one bacterial species, iv) sequencing the isolated nucleic acids, and v) comparing the sequenced nucleic acids to a reference nucleic acid sequence. When performing the methods necessitating genotyping, any genotyping assay can be used. For example, this can be done by sequencing the 16S or the 23 S ribosomal subunit or by metagenomics shotgun DNA sequencing associated with metatranscriptomics.

Methods for determining microbiome composition may include one or more microbiology methods such as sequencing, next generation sequencing, wester blotting, comparative genomic hybridization, PCR, ELISA, etc.

### VIII. KITS

Certain aspects of the disclosure also encompass kits for performing the methods of the disclosure, such as detection of, diagnosis of, or treatment of colitis and/or detection and qualitative or quantitative characterization of microorganisms. Such kits can be prepared from readily available materials and reagents. For example, such kits can comprise any one or more of the following materials: enzymes, reaction tubes, buffers, detergent, primers, probes, antibodies. In a preferred embodiment, these kits allow a practitioner to obtain samples of neoplastic cells in blood, tears, semen, saliva, urine, tissue, serum, stool, sputum, cerebrospinal fluid and supernatant from cell lysate. In another preferred embodiment these kits include the needed apparatus for performing RNA extraction, RT-PCR, and gel electrophoresis. Instructions for performing the assays can also be included in the kits.

In a particular aspect, these kits may comprise a plurality of agents for assessing or identifying microorganisms, wherein the kit is housed in a container. The kits may further comprise instructions for using the kit for assessing sequences, means for converting and/or analyzing sequence data to generate prognosis. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers of the invention. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

Further kit embodiments relate to kits comprising the therapeutic compositions of the disclosure. The kits may be useful in the treatment methods of the disclosure and comprise instructions for use.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Fecal Microbiota Transplantation for Refractory Immune Checkpoint Inhibitor-Associated Colitis

The inventors report the first case series of immune checkpoint inhibitors (ICI)-associated colitis successfully treated with fecal microbiota transplantation, with reconstitution of the gut microbiome and a relative increase in the proportion of regulatory T-cells within the colonic mucosa. These preliminary data provide evidence that modulation of the gut microbiome may abrogate ICI-associated colitis.

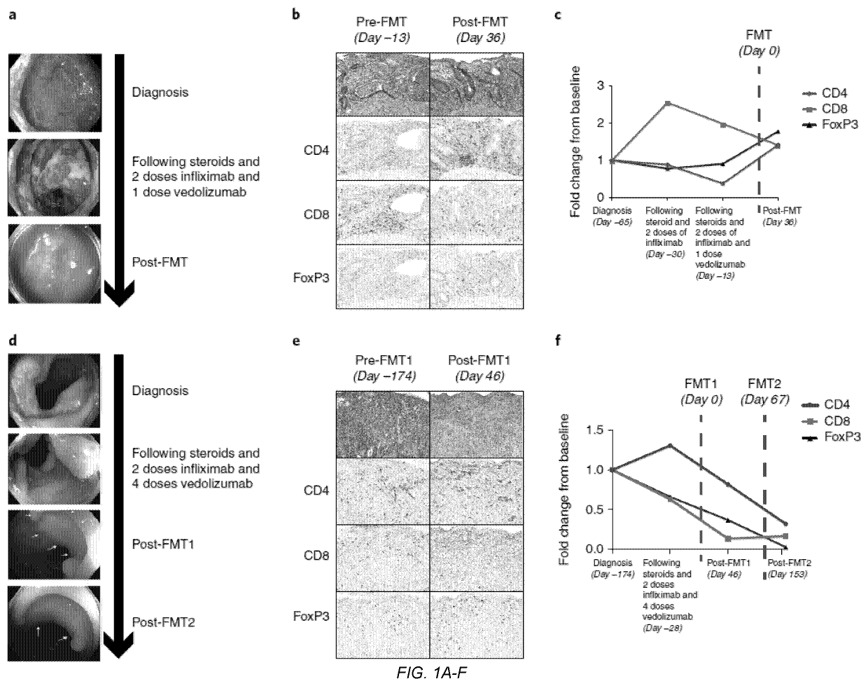

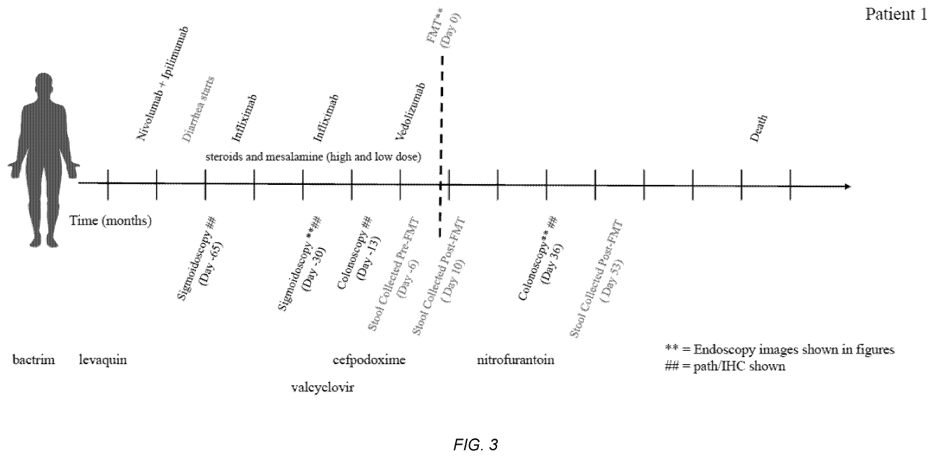

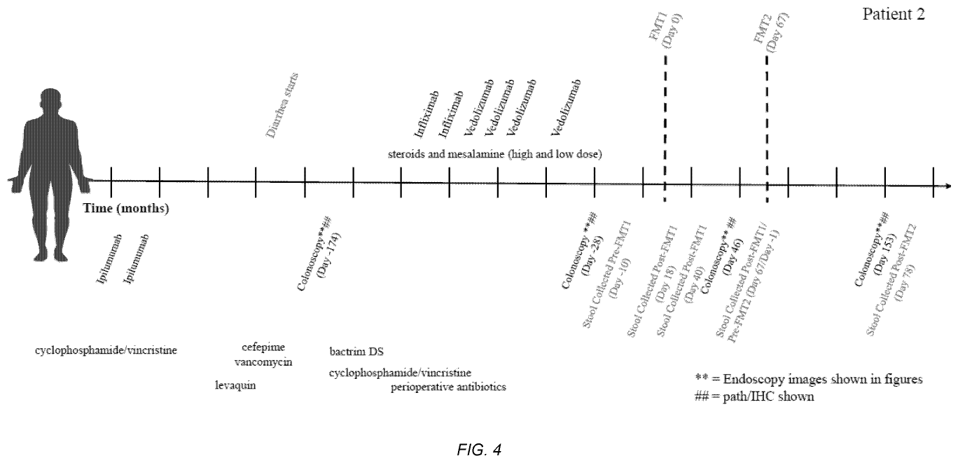

The inventors sought to determine the impact of treatment with FMT from healthy donors in patients with refractory ICI-associated colitis and enrolled two patients onto this treatment protocol between June 2017 and January 2018 (CIND17-0036, CIND17-0058). Clinical courses for both patients are further detailed in FIGS. 3-5. The first patient was a 50-year-old female with high-grade metastatic urothelial carcinoma refractory to standard chemotherapy who was enrolled onto a trial of combined CTLA-4 and PD-1 blockade (NCT1928394). Two weeks after treatment initiation, she was hospitalized with CTCAE Grade ≥2 diarrhea/colitis. An infectious workup including PCR-based multiplex assay for common gastrointestinal pathogens was negative, and colonoscopy demonstrated severe colitis that endoscopically resembled ulcerative colitis (FIG. 1A and FIGS. 5A and 6A). She received systemic corticosteroids, followed later by two doses of an anti-TNF-α agent (infliximab) as well as one dose of anti-integrin therapy (vedolizumab), but her symptoms persisted. She then received a single dose of FMT (50 g of donor stool) via colonoscopy. The second patient enrolled was a 78-year-old male with prostate cancer refractory to chemotherapy and hormonal therapy who received two doses of ipilimumab in the context of a clinical trial (NCT02113657). Three months after treatment initiation, he was hospitalized with fever and CTCAE Grade ≥2 diarrhea/colitis. Infectious etiologies were excluded and colonoscopy confirmed the diagnosis of ICI-associated colitis, although with a Crohn' s colitis-like presentation (FIGS. 1D, 5B, and 6B). His symptoms persisted despite systemic corticosteroids, infliximab, and vedolizumab. He received two doses of FMT. The source of all three FMT products was from a single healthy unrelated donor, collected at three different time points.

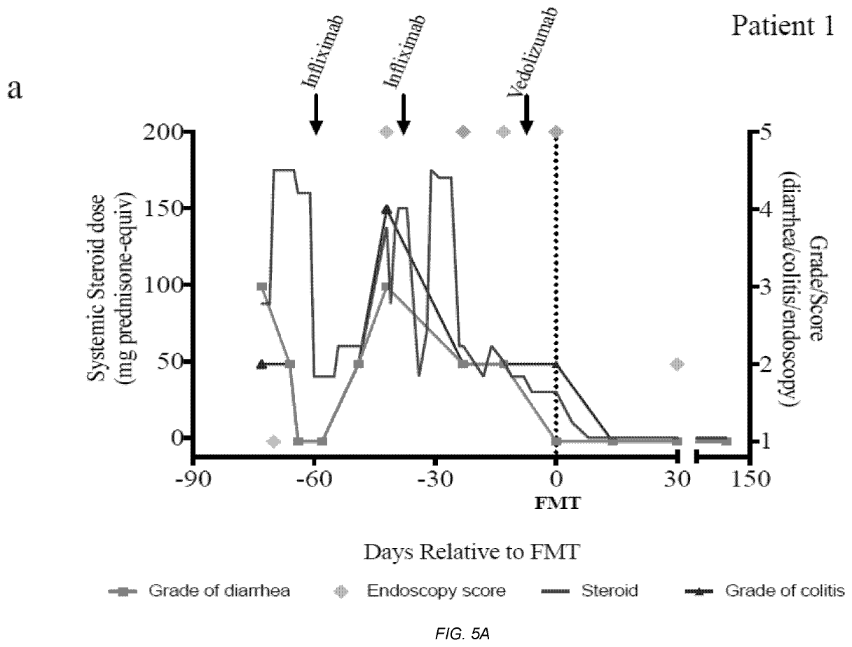

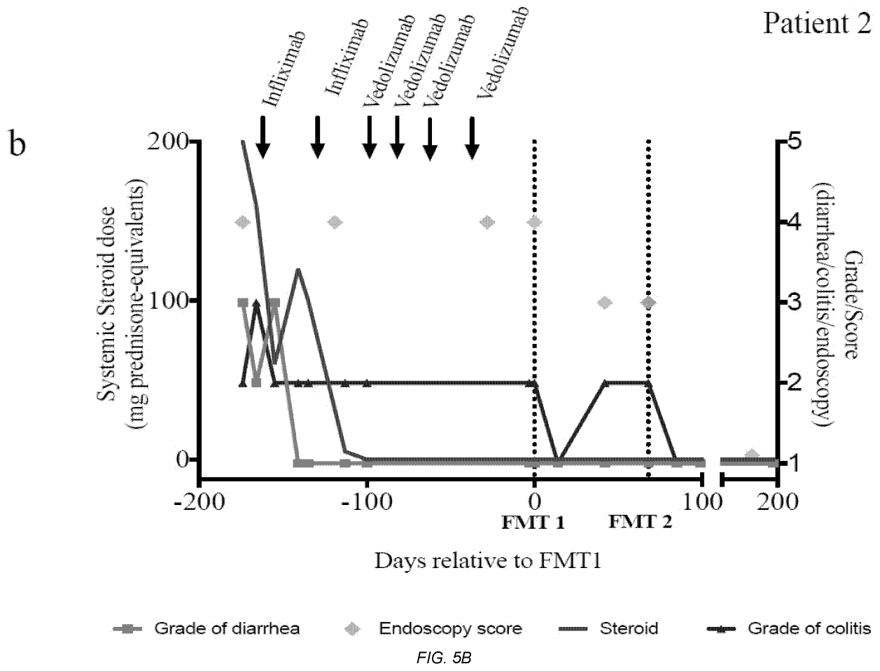

Both patients had complete resolution of clinical symptoms following treatment with FMT, with eventual return to normal solid daily bowel movements without further bleeding (FIG. 5). In the first patient, complete resolution occurred gradually within two weeks and she was weaned off steroids in seven days (FIG. 5A), while the second patient experienced partial improvement of gastrointestinal symptoms but with persistent ulcers on follow-up colonoscopy and recurrent abdominal pain. He experienced complete resolution after a second FMT treatment (FIG. 5B).

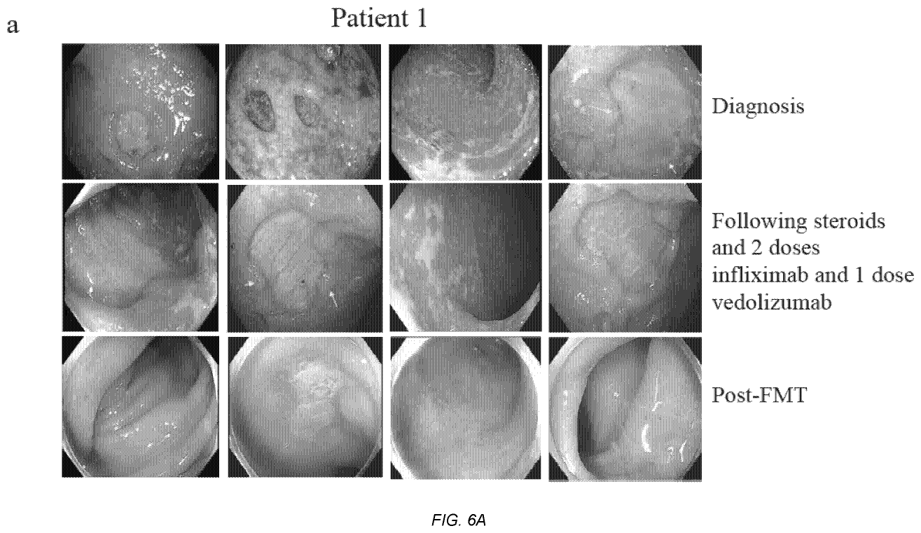

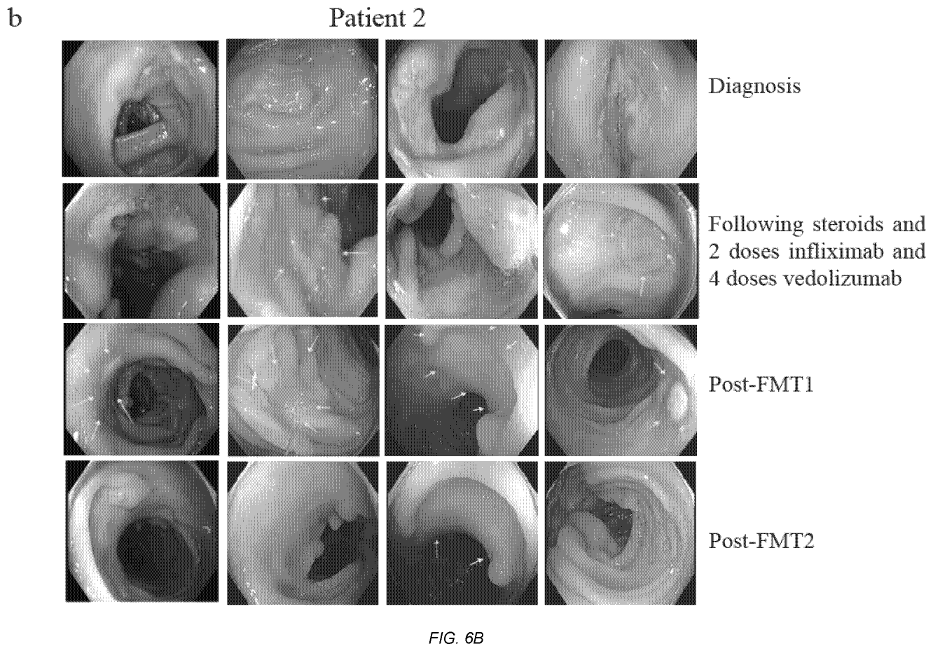

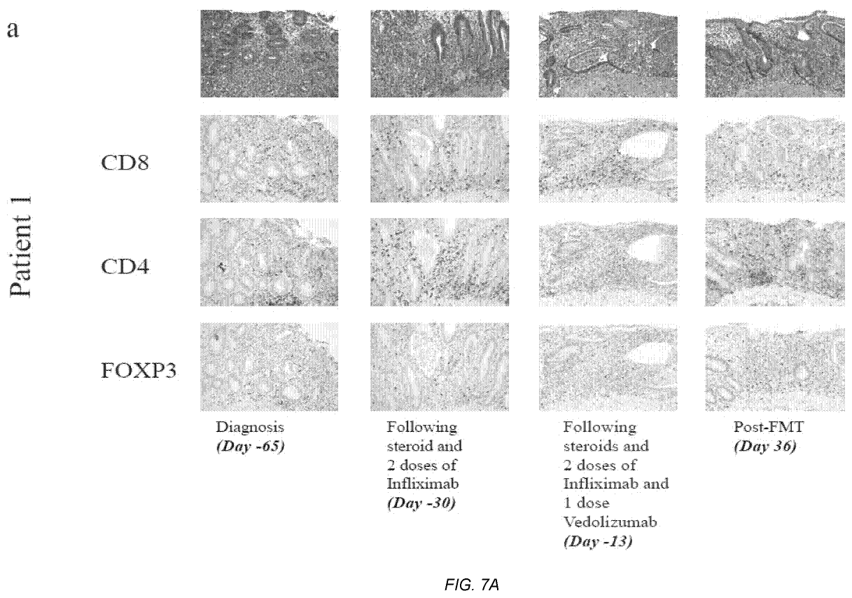

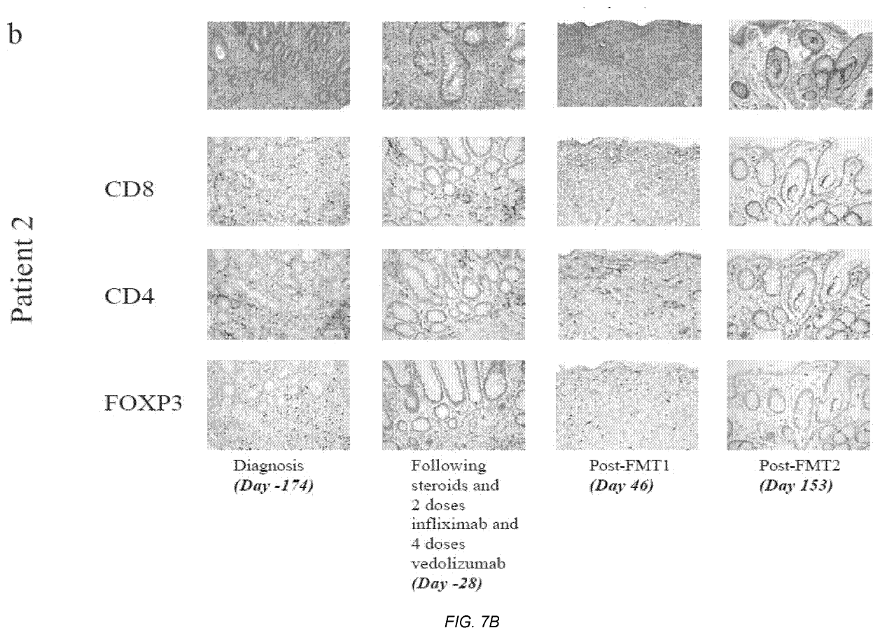

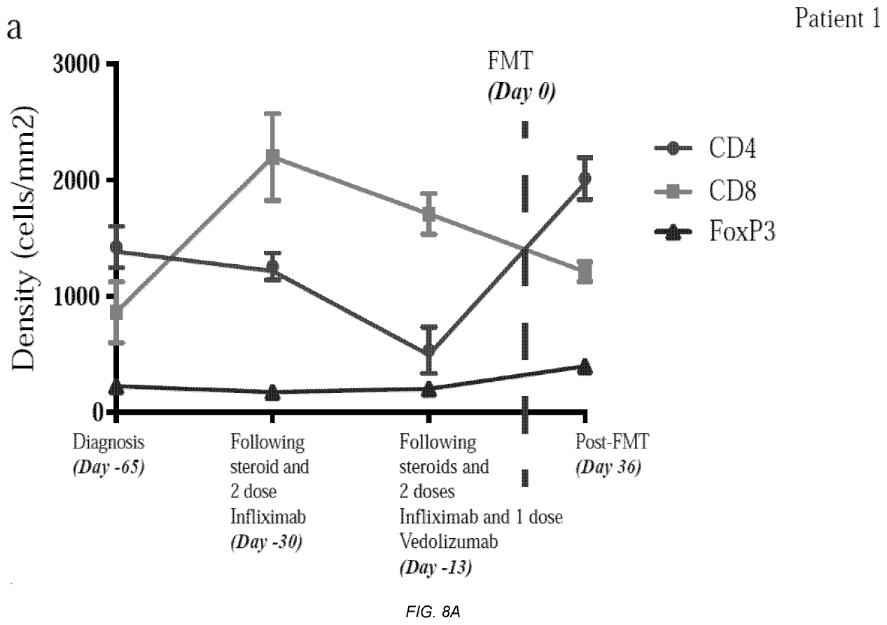

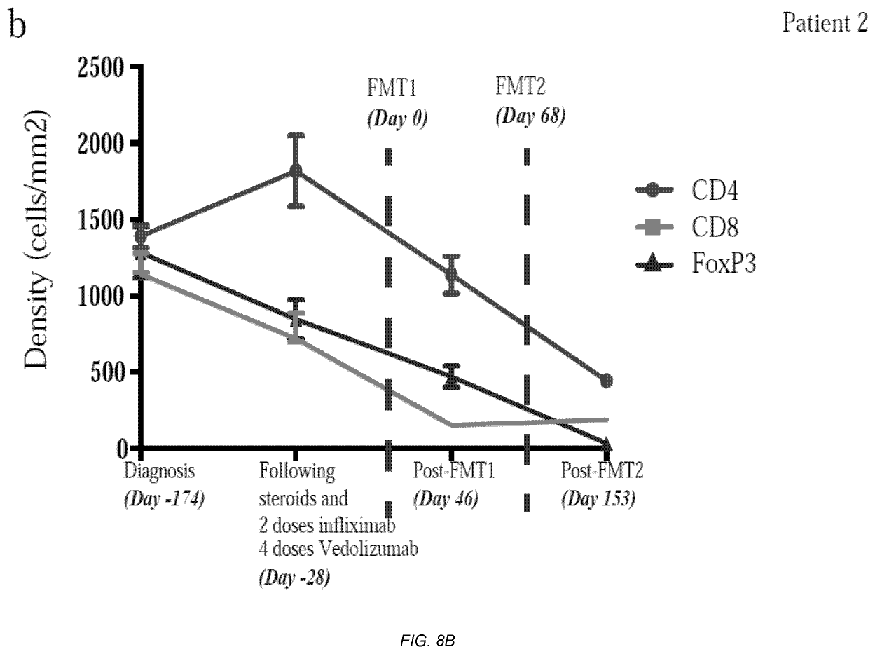

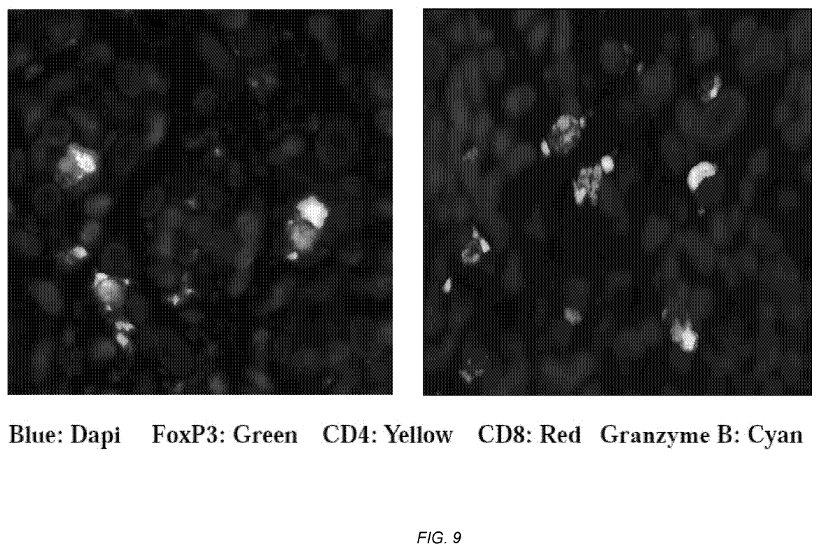

Endoscopic evaluation demonstrated significant mucosal inflammation and ulceration in both patients near the time of diagnosis of ICI-associated colitis, without substantial improvement after systemic corticosteroids, anti-TNF, and anti-integrin agents. Following FMT, marked improvement was evident on endoscopic evaluation, with reduced inflammation and resolution of ulcerations (FIGS. 1A, 1D, and 6). In the first patient, analysis of immune infiltrates in the colonic mucosa demonstrated a dense inflammatory infiltrate prior to FMT with a high density of CD8+ cytotoxic T lymphocytes and a low density of CD4+ FoxP3+ T cells (FIGS. 1B, 1C, 7A, and 8A), consistent with findings from reports of autoimmune colitis. Following FMT, there was a substantial reduction in CD8+ T-cell density with a concomitant increase in CD4+ FoxP3+ (FIGS. 1B, 1C, 7A, and 8A), offering a potential mechanism through which FMT could abrogate ICI-associated toxicity. In the second patient, the density of all T-cell subtypes analyzed decreased following FMT, but the CD4+ T-cell population was relatively spared compared to the CD8+ T-cell population, with persistence again noted for CD4+ and FoxP3+ cells (FIGS. 1E, 1F, 7B, 8B, and 9).

Endoscopic evaluation demonstrated significant mucosal inflammation and ulceration in both patients near the time of diagnosis of ICI-associated colitis, without substantial improvement after systemic corticosteroids, anti-TNF, and anti-integrin agents. Following FMT, marked improvement was evident on endoscopic evaluation, with reduced inflammation and resolution of ulcerations (FIGS. 1A, 1D, and 6). In the first patient, analysis of immune infiltrates in the colonic mucosa demonstrated a dense inflammatory infiltrate prior to FMT with a high density of CD8+ cytotoxic T lymphocytes and a low density of CD4+ FoxP3+ T cells (FIGS. 1B, 1C, 7A, and 8A), consistent with findings from reports of autoimmune colitis. Following FMT, there was a substantial reduction in CD8+ T-cell density with a concomitant increase in CD4+ FoxP3+ (FIGS. 1B, 1C, 7A, and 8A), offering a potential mechanism through which FMT could abrogate ICI-associated toxicity. In the second patient, the density of all T-cell subtypes analyzed decreased following FMT, but the CD4+ T-cell population was relatively spared compared to the CD8+ T-cell population, with persistence again noted for CD4+ and FoxP3+ cells (FIGS. 1E, 1F, 7B, 8B, and 9).

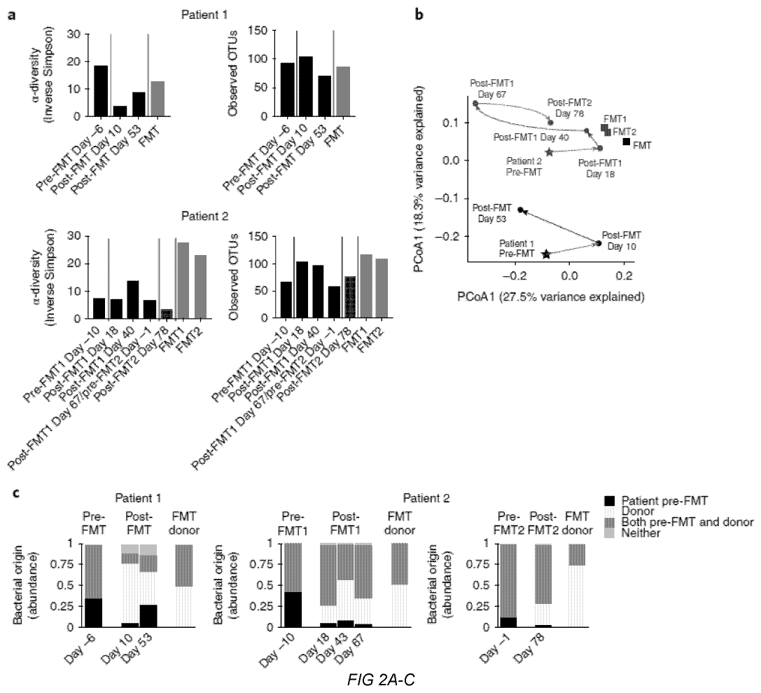

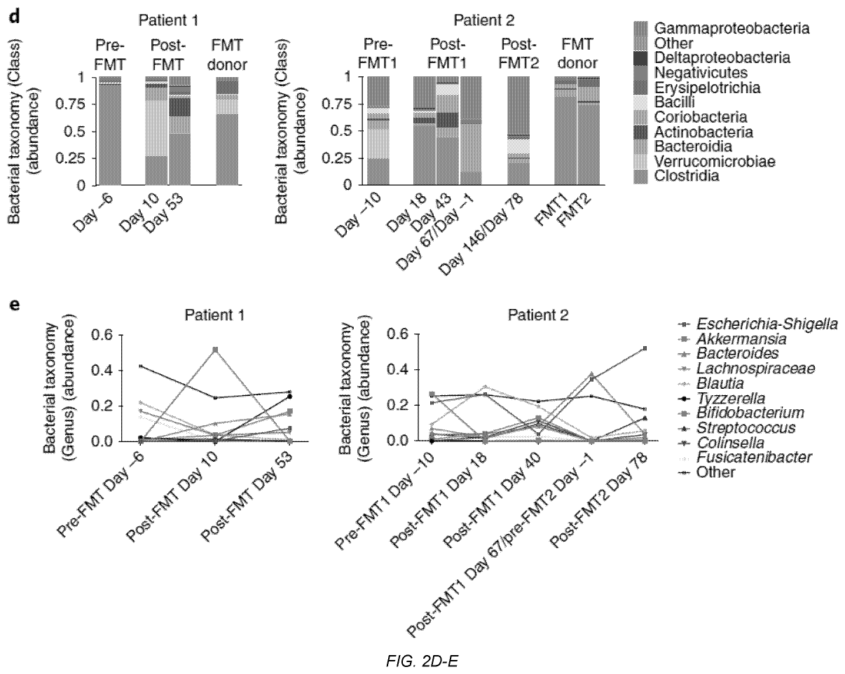

The inventors next assessed bacterial taxa present at time of colitis in these patients as well as compositional changes in the gut microbiome following treatment with FMT. Bacterial taxa present at time of colitis were quite disparate between the two patients, with a predominance of Clostridia and a notable absence of bacteria shown to be protective against ICI-associated colitis and IBD such as Bacteroidia and Verrucomicrobiae, respectively, in the first patient and a predominance of Gammaproteobacteria (predominantly Escherichia) in the second patient, which is commonly seen in perturbed intestinal states. Immediately following FMT in the first patient, donor FMT-derived bacteria had effectively colonized the intestinal tract, with nearly 75% of the sequences uniquely attributable to the FMT donor microbiome and a notably higher abundance of Akkermansia (FIGS. 2D and 2E). By week 7 after FMT, Akkermansia now only accounted for a small portion of her microbiome and there was further expansion of Clostridia, which were largely patient-derived in origin (FIG. 2D, left and 2E, left). Of note, the patient also showed an expansion of Bifidobacterium after FMT, which recently was reported to abrogate ICI-related toxicity in a murine model (FIG. 2D, left and 2E, left). In the second patient, there was a notable increase in the abundance of Blautia and Bifidobacterium species after FMT, which have been associated with reduced intestinal inflammation (FIG. 2D, right and 2E, right). In addition to this, he had a decrease in the abundance of potentially pathogenic Escherichia and an increase in potentially beneficial Bacteroides after his first FMT (FIG. 2D, right and 2E, right). After his second FMT, he had a higher abundance of Escherichia and an eventual decrease in Bacteroides; however, his gastrointestinal symptoms steadily continued to improve (FIG. 2D, right and 2E, right).

Together, these cases provide provocative and novel evidence that modulation of the gut microbiome via FMT can be associated with significant and rapid improvement of refractory ICI-associated colitis with early insights into potential mechanisms.

A. Methods

Donor selection stool bank is covered by the institutional review board HSC-SPH-15-0991 at the University of Texas School of Public Health. A single anonymous donor provided the fecal stool employed in this study after appropriate screening (Tables 1 and 2).

1. Donor Stool Preparation

Individual stool samples from the donor of ≥150 g were processed within 4 h of passage, diluted in 0.85% NaCl (1:10) with a total volume of 1,500 ml, mixed in a StomacherR 80 Master (Seward Laboratory System) in a sterilized bag, and then filtered through moistened five-layer sterile gauze in a funnel (both sterilized) within a biological safety cabinet. Frozen aliquots were stored at −80° C. and used within 6 months of preparation. On the day of FMT, 250 ml of frozen product was dissolved and packaged into five 50 ml sterilized syringes. After reconstitution, the product was kept at 4° C. and used within 4 h.

2. Delivery of FMT

As a standard procedure, colonoscopy was performed on the recipient at MD Anderson's Endoscopy Unit following an overnight colonic cleansing preparative regimen. Once the scope reached the cecum, 50 g/250 ml of liquid donor stool was delivered through the water channel of the scope to the cecum by preloaded syringes. Recipients rested in bed for 1 h post procedure before discharge from the endoscopy unit. Patients were instructed to resume their normal daily routines, including diet, following the FMT procedure.

3. Patient Clinical Histories

Patient 1 was a 50-year-old female with high-grade metastatic urothelial carcinoma metastatic to the lung and spine refractory to standard chemotherapy regimens. She was enrolled onto a trial of combined ipilimumab and nivolumab blockade (NCT1928394). Two weeks after treatment initiation, she was hospitalized with CTCAE (common terminology criteria for adverse events) Grade ≥2 diarrhea/colitis (bloody diarrhea) diagnosed clinically and confirmed with endoscopy after infectious etiologies were excluded. Her symptoms persisted despite standard therapies for colitis. This included 3 months of ongoing systemic corticosteroids with two doses of an anti-TNF-α agent (infliximab) during a prolonged hospitalization. Cytomegalovirus immunohistochemistry appeared positive on histology obtained at subsequent endoscopy; she was successfully treated with valcyclovir with resolution on follow-up endoscopic biopsy. As third-line therapy, one dose of anti-integrin antibody (vedolizumab) was given without further improvement. As compassionate treatment (CIND17-0036, IRB PA18-0372), she received a single dose of FMT (50 g of donor stool) via colonoscopy. The patient improved clinically. However, she eventually succumbed to progression of her primary cancer 3 months after FMT. Patient 2 was a 78-year-old male with prostate cancer metastatic to bone refractory to chemotherapy and hormonal therapy, who received two doses of ipilimumab in the context of a clinical trial (NCT02113657). Three months after treatment initiation, he was hospitalized with fever and CTCAE Grade≥2 diarrhea/colitis (diarrhea, rectal bleeding, and abdominal pain) diagnosed clinically and confirmed with endoscopy after infectious etiologies were excluded. He exhibited incomplete clinical, endoscopic, and histological improvement on standard therapies for colitis including a total of 5 months of immunosuppressive agents (systemic corticosteroids, two doses of infliximab, and four doses of vedolizumab). As compassionate treatment (CIND17-0058, IRB PA18-0372), he received FMT treatment (also 50 g of donor stool via colonoscopy), which improved his symptoms in the short term; however, multiple colonic ulcerations persisted with residual abdominal pain. He received a second dose of FMT (50 g of donor stool via colonoscopy) 2 months later under the same CIND17-0058. He has remained asymptomatic for 7 months thus far, although he has since received additional cancer treatments.

4. Methods for Microbiome Analysis

Microbiome analysis was performed on the patient samples collected pre-FMT and on days 10 and 53 after FMT, and on samples collected from the FMT donor. Genomic bacterial DNA was extracted from fecal samples using the QIAamp DNA Stool kit (Qiagen), with the addition of a bead-beating lysis step. Genomic 16S ribosomal-RNA V4 variable regions were amplified and sequenced on the Illumina MiSeq platform as previously described24. Between 3,380 and 42,776 sequences were obtained for each sample (average 10,003).

VSEARCH was used for analyzing nucleotide sequences. Paired-end reads were merged, de-replicated, and sorted by length and size. Sequences were then error-corrected and chimera-filtered using the UNOISE algorithm (available on the world wide web atbiorxiv.org/content/early/2016/10/15/081257) to generate a preliminary list of OTUs. Both OTUs and presumed chimeras were assigned taxonomy in QIIME26 using the Mothur method with the Silva database version 128 available on the world wide web at databasecommons.org/database.jsp?db_id=238). Additionally, sequences rejected by the UNOISE algorithm that matched a database entry with a perfect score were restored to generate the final list of OTUs. An OTU table was generated using VSEARCH, and UniFrac distances between samples were determined with QIIME. For assessment of the inverse Simpson's diversity score, sample sequences were first rarefied at a number below the sample with the least number of sequences (3,000) using QIIME. In addition, principal coordinate analysis (PCoA) was used to illustrate the unweighted UniFrac distance between study patients and donor samples in two dimensions PC1 and PC2 to construct an orthogonal coordinate that displays the most variation between samples.

5. Methods for Immunohistochemistry

Sections (4 μm thickness) were prepared from formalin-fixed paraffin-embedded gut tissues. Slides were then stained using a Leica Bond RX automated slide stainer (Leica Biosystems) for CD3 (1:100, Dako), CD8 (1:100, Thermo Scientific), and FoxP3 (1:50, BioLegend), and counter-stained with hematoxylin. Stained slides were then scanned using an automated Aperio Slide Scanner (Leica), and the density of the immune infiltrate was quantified in tumor regions using a modified version of the default ‘Nuclear v9’ algorithm and expressed as positive counts per mm².

For each immunohistochemical (IHC) marker, the inventors assessed a single 5 mm section. For the automated image analyses, four regions of interest (ROI) were manually selected from each 5 mm section per marker per sample. Each of the four ROIs measured 0.5×0.5 mm², and the density of IHC+ cells plotted for each time point represents the mean of the four ROIs per mm². Absolute densities of immune cells are provided with the measure of variance (standard deviation).

6. Methods for Multiplex Immunofluorescence Assay and Multispectral Analysis

For multiplexed staining, the inventors followed the Opal protocol staining method for the following markers: CD4 (1:25, CM153BK, Biocare) with subsequent visualization accomplished using fluoresceinAF-647; FoxP3 with visualization using AF-488 (1:50); CD8 (1:200, M7103, Dako) with visualization using AF-594 (1:50); and Granzyme B (1:100, PA0291, Leica Microsystems) with visualization using AF-555 (1:50). Nuclei were subsequently visualized with DAPI (1:2,000). All of the sections were cover-slipped using Vectashield Hardset 895 mounting media.

A detailed methodology for multispectral analysis is described previously. Each of the individually stained sections (CD4/AF-647, CD8/AF-594, FoxP3/AF-488, GrB/AF-555, and DAPI) were utilized to establish the spectral library of fluorophores required for multispectral analysis. The slides were scanned using the Vectra slide scanner (PerkinElmer) under fluorescent conditions. For each marker, the mean fluorescent intensity per case was then determined as a base point from which positivity calls could be established as a positivity threshold.

7. Methods of Stool Analysis for Infectious Agents

Real-time PCR methodologies were performed to detect the presence of nucleic acid from the following 22 pathogens: Adenovirus F 40/41, Astrovirus, C. difficile, Campylobacter, Cryptosporidium, Cyclospora cayetanensis, Escherichia coli 0157, Enteroaggregative E. coli, Enteroinvasive E. coli, Entamoeba histolytica, Enteropathogenic E. coli, Enterotoxigenic E. coli, Giardia lamblia, Norovirus GI/GII, Plesiomonas shigelloides, Rotavirus A, Salmonella, Sapovirus (I, II, IV, and V), Shiga toxin-producing E. coli, Vibrio, Vibrio cholera, Yersinia enterocolitica. Positive results were confirmed with either culture for most agents or ELISA for C. difficile.

8. Methods for Endoscopic Scoring for ICI-Associated Colitis.

Both patients had full colonoscopic evaluations that examined every segment of the colon (ascending, transverse, descending, and sigmoid colon, and rectum). Given the qualitative nature of endoscopic data collection and an inability to provide true statistical analyses, other multiple representative photographs from the same colonoscopic evaluations were included.

An endoscopic severity score specific to ICI-associated colitis that incorporates the presence of erythema and ulcerations as well as the number and depth of mucosal ulcerations was utilized. The endoscopy score consists of five characteristics with one point awarded for each of the following: (a) erythema and erosions; (b) any ulcer; (c) more than two ulcers in number; (d) ulcers larger than 1 cm surface area; and (e) ulcers deeper than 2 mm.

9. Statistical Treatment of Data and Analyses

For qualitative data including endoscopy, the inventors have included multiple representative photographs as discussed above. For the IHC, the inventors chose a single slide from each patient for each time point to stain but have included multiple time points per patient. Stained slides were then scanned using an automated Aperio Slide Scanner (Leica), and the density of the immune infiltrate was quantified in tumor regions using a modified version of the default ‘Nuclear v9’ algorithm and expressed as positive counts per mm². For each IHC marker, the inventors assessed a single 5 mm section. For the automated image analyses, four ROIs were manually selected for each 5 mm section per marker per sample. Each of the four ROIs measured 0.5×0.5 mm², and the density of IHC+ cells plotted for each.

The time point represents the mean of the four ROIs per mm². Absolute densities of immune cells are provided with the measure of variance (standard deviation). For the multiplex IHC, the inventors' objective was not to quantitate the number of different cell types within each sample but rather to demonstrate colocalization of CD4 and FoxP3. Thus, the inventors chose a representative slide from both patients and multiple (>2 time points) were assessed. Regarding the stool analyses, a single sample from each patient at each time point was analyzed. As noted above, between 3,380 and 42,776 sequences were obtained for each sample (average 10,003).

TABLE 1
Donor Screening Tests

| | | Acceptance |
|---|---|---|
| Agent | Material | Criteria |
| Hepatitis B Core Antibody | Blood | Negative |
| Hepatitis B Surface Antigen | Blood | Negative |
| Hepatitis C Virus Antibody | Blood | Negative |
| Hepatitis A Virus IgM | Blood | Negative |
| HIV-1 and HIV-2 Antibody | Blood | Negative |
| Anti-HTLV I/II | Blood | Negative |
| Serologic Test for Syphilis | Blood | Negative |
| Clostridium difficile toxin A/B | Stool | Negative |
| Shigella spp. | Stool | Negative |
| Salmonella spp. | Stool | Negative |
| Campylobacter spp. | Stool | Negative |
| Shiga-toxin producing Escherichia coli | Stool | Negative |
| Methicillin Resistant Staphylococcus aureus | Stool | Negative |
| Vancomycin Resistant Enterococcus spp. | Stool | Negative |
| Carbapenem Resistant Enterobacteriacea | Stool | Negative |
| Extended Spectrum b-lactanase Producing E. coli | Stool | Negative |
| Aeromonas spp. | Stool | Negative |
| Plesiomonas spp. | Stool | Negative |
| Yersinia spp. | Stool | Negative |
| Vibrio spp. | Stool | Negative |
| Cryptosporidum | Stool | Negative |
| Entamoeba histolytica | Stool | Negative |
| Cyclospora | Stool | Negative |
| Isospora | Stool | Negative |
| Rotavirus | Stool | Negative |
| Adenovirus | Stool | Negative |
| Norovirus | Stool | Negative |
| Giardia lamblia, EIA | Stool | Negative |
| H. Pylori EIA | Stool | Negative |

TABLE 2
Donor Screening Tests

| Inclusion | Exclusion |
|---|---|
| 1. Must be ≥18 years of age | 1. Tested positive for any of variables mentioned |
| 2. Able to provide and sign informed | below |
| consent | 2. History of autoimmune or atopic illness or active |
| 3. Able to complete and sign the donor | cancer or ongoing immune modulating therapy |
| questionnaire | 3. First degree relative with intestinal carcinoma |
| 4. Able to adhere to fecal | 4. History of risk factors for acquisition of HIV, |
| transplantation stool collection | syphilis, Hepatitis B, Hepatitis C, prion or any |
| requirements | neurological disease as determined by the donor |
| | questionnaire |
| | 5 History of gastrointestinal disorder, e.g., |
| | inflammatory bowel disease (IBD), irritable bowel |
| | syndrome (IBS), chronic constipation or diarrhea, |
| | gastrointestinal malignancies |
| | 6. New sexual contacts during past 6 months |
| | 7. Tattoos, body piercing or incarceration within 6 |
| | months |
| | 8 Major gastrointestinal surgical procedures |
| | 9. Antibiotic use during the preceding 3 months of |
| | donation |
| | 10. Drug or alcohol abuse |
| | 11. Fever >100.4° F. (38° C.) for the past 3 months |
| | 12. Signs or any symptoms, including persistent |
| | symptoms of communicable infection, including cold |
| | 13. A history of chronic pain syndromes |
| | (fibromyalgia, chronic fatigue) or neurologic, |
| | neurodevelopmental disorders |
| | 14. Receipt of any type of live vaccine within 3 |
| | months prior to stool donation |
| | 15. Current or previous medical or psychosocial |
| | condition |
| | 16. Metabolic syndrome, body mass index over 30 or |
| | moderate-to-severe undernutrition (Malnutrition) |
| | 17. Hospitalization during the preceding 3 months of |
| | donation |
| | 18. Regular attendance at outpatient medical or |
| | surgical clinics |
| | 19. International travel or recent medical tourism |
| | within 3 months periodRegular attendance at |
| | outpatient medical or surgical clinics |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

1. Michot, J. M. et al. Eur. J. Cancer 54, 139-148 (2016).

2. Cramer, P. & Bresalier, R. S. Curr. Gastroenterol. Rep. 19, 3 (2017).

3. Chen, J. H., Pezhouh, M. K., Lauwers, G. Y. & Masia, R. Am. J. Surg. Pathol. 41, 643-654 (2017).

4. Dadu, R., Zobniw, C. & Diab, A. Cancer J. 22, 121-129 (2016).

5. Bertrand, A., Kostine, M., Barnetche, T., Truchetet, M. E. & Schaeverbeke, T. BMC Med. 13, 211 (2015).

6. Horvat, T. Z. et al. J. Clin. Oncol. 33, 3193-3198 (2015).

7. Beck, K. E. et al. J. Clin. Oncol. 24, 2283-2289 (2006).

8. Johnston, R. L., Lutzky, J., Chodhry, A. & Barkin, J. S. Dig. Dis. Sci. 54, 2538-2540 (2009).

9. Minor, D. R., Chin, K. & Kashani-Sabet, M. Cancer Biother. Radiopharm. 24, 321-325 (2009).

10. Borody, T. J. & Khoruts, A. Nat. Rev. Gastroenterol. Hepatol. 9, 88-96 (2011).

11. Gopalakrishnan, V. et al. Science 359, 97-103 (2018). 1

12. Matson, V. et al. Science 359, 104-108 (2018).

13. Routy, B. et al. Science 359, 91-97 (2018).

14. Dubin, K. et al. Nat. Commun. 7, 10391 (2016). 1

15. Vetizou, M. et al. Science 350, 1079-1084 (2015).

16. Chaput, N. et al. Ann. Oncol. 28, 1368-1379 (2017).

17. Wang, F., Yin, Q., Chen, L. & Davis, M. M. Proc. Natl Acad. Sci. USA 115, 157-161 (2018).

18. Kappeler, A. & Mueller, C. Histol. Histopathol. 15,167-172 (2000).

19. Nancey, S. et al. Gastroenterology 131,485-496 (2006).

20. Png, C. W. et al. Am. J. Gastroenterol. 105,2420-2428 (2010).

21. Litvak, Y., Byndloss, M. X., Tsolis, R. M. & Baumler, A. J. Curr. Opin. Microbiol. 39,1-6 (2017).

22. Jenq, R. R. et al. Biol. Blood Marrow Transplant. 21,1373-1383 (2015).

23. van Nood, E. et al. N. Engl. J Med. 368, 407-415 (2013).

24. Caporaso, J. G. et al. ISMEJ. 6, 1621-1624 (2012)

25. Rognes, T., Flouri, T., Nichols, B., Quince, C. & Mahe, F. PeerJ 4, e2584 (2016).

26. Caporaso, J. G. et al. Nat. Methods 7,335-336 (2010).

27. Schloss, P. D. et al. Appl. Environ. Microbiol. 75,7537-7541 (2009).

28. Lozupone, C., Lladser, M. E., Knights, D., Stombaugh, J. & Knight, R. ISMEJ 5, 169-172 (2011).

29. Stack, E. C., Wang, C., Roman, K. A. & Hoyt, C. C. Methods 70, 46-58 (2014).

30. Abu-Sbeih, H. et al. J. Immunother. Cancer 6, 1-11 (2018).

1. A method for treating immune checkpoint inhibitor (ICI)-associated colitis in a subject comprising administering fecal matter from a healthy donor to the subject.

2. The method of claim 1, wherein the ICI-associated colitis comprises refractory ICI-associated colitis.

3. The method of claim 1 or 2, wherein the subject has been treated with anti-CTLA-4 monotherapy.

4. The method of claim 1 or 2, wherein the subject has been treated with anti-PD-1 monotherapy.

5. The method of claim 1 or 2, wherein the subject has been treated with anti-CTLA-4 and anti-PD-1 combination therapy.

6. The method of any one of claims 1-5, wherein the colitis is classified as a Grade 2 or greater.

7. The method of any one of claims 1-6, wherein the subject has received a previous treatment for the ICI-associated colitis.

8. The method of claim 7, wherein the subject has been determined to be unresponsive to the previous treatment.

9. The method of claim 7 or 8, wherein the previous treatment comprises one or more of steroids, corticosteroids, anti-TNF-alpha therapy, anti-integrin therapy, infliximab, mesalamine, and vedolizumab.

10. The method of claim 9, wherein the steroid comprises methylprednisolone or prednisolone.

11. The method of claim 10, wherein the subject has been determined to be unresponsive to intravenous methylprednisolone 140 mg/day for at least 5 days.

12. The method of any one of claims 8-11, wherein the subject has been determined to be unresponsive or further unresponsive to at least one dose of 5 mg/kg of infliximab.

13. The method of any one of claims 8-12, wherein the subject has been determined to be unresponsive or further unresponsive to intravenous methylprednisolone 110 mg/day for at least 2 days.

14. The method of any one of claims 8-13, wherein the subject has been determined to be unresponsive or further unresponsive to a dose of 10 mg/kg infliximab.

15. The method of any one of claims 1-14, wherein the method comprises the administration of at least 2 doses of fecal matter.

16. The method of claim 15, wherein the two administrations are at least 30 days apart.

17. The method of any one of claims 1-16, wherein the administration comprises intracolonic administration.

18. The method of claim 17, wherein the administration comprises intracolonic administration to the cecum.

19. The method of any one of claims 1-17, wherein the method further comprises administration of one or more treatments.

20. The method of claim 19, wherein the one or more treatments comprises one or more of corticosteroids, anti-TNF-alpha therapy, anti-integrin therapy, infliximab, mesalamine, and vedolizumab.

21. The method of any one of claims, wherein the method excludes one or more additional treatments after, at the most, 30 days post fecal matter administration.

22. The method of claim 21, wherein the method excludes administration of steroids after 30 days post fecal matter administration.

23. The method of any one of claims 1-22, wherein the healthy donor does not have cancer or has not been previously treated for cancer.

24. The method of any one of claims 1-23, wherein the healthy donor does not have colitis.

25. The method of any one of claims 1-24, wherein the subject has been diagnosed with refractory cancer.

26. The method of any one of claims 1-25, wherein the subject was administered immune checkpoint inhibitor therapy prior to administration of the fecal matter.

27. The method of any one of claims 1-26, wherein the subject is currently undergoing an immune checkpoint inhibitor therapy regimen.

28. The method of claim 26, wherein the administration of the immune checkpoint therapy and the fecal matter occurs within 7 days.

29. The method of any one of claims 1-28, wherein the fecal matter is administered in a dose of 50 g.

30. The method of anyone of claims 1-29, wherein the administration provides for a reduction in CD8+ T-cell density or in CD8+ cytotoxic T lymphocytes.

31. The method of anyone of claims 1-30, wherein the administration provides for an increase in CD4+ FoxP3+ T cells.

32. A method of treating immune checkpoint inhibitor (ICI)-associated colitis in a subject comprising administering to the subject a composition comprising at least one isolated or purified population of bacteria belonging to one or more of the genera Escherichia, Akkermansia, Bacteroides, Lachnospiraceae, Blautia, Tyzzerella, Bifidobacterium, Streptococcus, Colinsella, and Fusicatenibacter.

33. The method of claim 32, wherein the composition comprises at least one isolated or purified population of bacteria belonging to one or more of the genera Akkermansia, Blautia, Bifidobacterium, Bacteroides, and Escherichia.

34. The method of claim 33, wherein Escherichia comprises Escherichia shigella.

35. The method of any one of claims 32-34, wherein the ICI-associated colitis comprises refractory ICI-associated colitis.

36. The method of any one of claims 32-35, wherein the subject has been treated with anti-CTLA-4 monotherapy.

37. The method of any one of claims 32-35, wherein the subject has been treated with anti-PD-1 monotherapy.

38. The method of any one of claims 32-35, wherein the subject has been treated with anti-CTLA-4 and anti-PD-1 combination therapy.

39. The method of any one of claims 32-38, wherein the colitis is classified as a Grade 2 or greater.

40. The method of any one of claims 32-39, wherein the subject has received a previous treatment for the ICI-associated colitis.

41. The method of claim 40, wherein the subject has been determined to be unresponsive to the previous treatment.

42. The method of claim 40 or 41, wherein the previous treatment comprises one or more of steroids, corticosteroids, anti-TNF-alpha therapy, anti-integrin therapy, infliximab, mesalamine, and vedolizumab.

43. The method of claim 42, wherein the steroid comprises methylprednisolone or prednisolone.

44. The method of claim 43, wherein the subject has been determined to be unresponsive to intravenous methylprednisolone 140 mg/day for at least 5 days.

45. The method of any one of claims 41-44, wherein the subject has been determined to be unresponsive or further unresponsive to at least one dose of 5 mg/kg of infliximab.

46. The method of any one of claims 41-45, wherein the subject has been determined to be unresponsive or further unresponsive to intravenous methylprednisolone 110 mg/day for at least 2 days.

47. The method of any one of claims 41-46, wherein the subject has been determined to be unresponsive or further unresponsive to a dose of 10 mg/kg infliximab.

48. The method of any one of claims 32-47, wherein the method comprises the administration of at least 2 doses of fecal matter.

49. The method of claim 48, wherein the two administrations are at least 30 days apart.

50. The method of any one of claims 32-49, wherein the administration comprises intracolonic administration.

51. The method of claim 50, wherein the administration comprises intracolonic administration to the cecum.

52. The method of any of claims 32-49, wherein the administration comprises oral administration and the composition is formulated for oral delivery.

53. The method of claim 52, wherein the composition formulated for oral delivery is a tablet or capsule.

54. The method of claim 53, wherein the tablet or capsule comprises an acid-resistant enteric coating.

55. The method of any one of claims 32-54, wherein the method further comprises administration of one or more treatments.

56. The method of claim 55, wherein the one or more treatments comprises one or more of corticosteroids, anti-TNF-alpha therapy, anti-integrin therapy, infliximab, mesalamine, and vedolizumab.

57. The method of any one of claims, wherein the method excludes one or more additional treatments after, at the most, 30 days post fecal matter administration.

58. The method of claim 57, wherein the method excludes administration of steroids after 30 days post fecal matter administration.

59. The method of any one of claims 32-58, wherein the healthy donor does not have cancer or has not been previously treated for cancer.

60. The method of any one of claims 32-59, wherein the healthy donor does not have colitis.

61. The method of any one of claims 32-60, wherein the subject has been diagnosed with refractory cancer.

62. The method of any one of claims 32-61, wherein the subject was administered immune checkpoint inhibitor therapy prior to administration of the fecal matter.

63. The method of any one of claims 32-62, wherein the subject is currently undergoing an immune checkpoint inhibitor therapy regimen.

64. The method of claim 62, wherein the administration of the immune checkpoint therapy and the fecal matter occurs within 7 days.

65. The method of any one of claims 32-64, wherein the fecal matter is administered in a dose of 50 g.

66. The method of anyone of claims 32-65, wherein the administration provides for a reduction in CD8+ T-cell density or in CD8+ cytotoxic T lymphocytes.

67. The method of anyone of claims 32-66, wherein the administration provides for an increase in CD4+ FoxP3+ T cells.

68. A composition comprising at least one isolated or purified population of bacteria belonging to one or more of the genera Escherichia, Akkermansia, Bacteroides, Lachnospiraceae, Blautia, Tyzzerella, Bifidobacterium, Streptococcus, Colinsella, and Fusicatenibacter.

69. A composition comprising at least two isolated or purified population of bacteria belonging to one or more of the genera Escherichia, Akkermansia, Bacteroides, Lachnospiraceae, Blautia, Tyzzerella, Bifidobacterium, Streptococcus, Colinsella, and Fusicatenibacter.

70. The composition of claim 68 or 69, wherein each of the populations of bacteria is present in the composition at a concentration of at least 10\hat{ }3 CFU.

71. The composition of any one of claims 68-70, wherein the composition is a live bacterial product or a live biotherapeutic product.

72. The composition of any one of claims 68-71, wherein the bacteria are lyophilized, freeze dried, or frozen.

73. The composition of any one of claims 68-72, wherein the composition is formulated for oral delivery.

74. The composition of claim 73, wherein the composition formulated for oral delivery is a tablet or capsule.

75. The composition of claim 74, wherein the tablet or capsule comprises an acid-resistant enteric coating.

76. The composition of any one of claims 68-72, wherein the composition comprising the at least one isolated or purified population of bacteria or the at least two isolated or purified populations of bacteria is formulated for administration rectally, via colonoscopy, sigmoidoscopy by nasogastric tube, or enema.

77. The composition of any one of claims 68-76, wherein the composition is capable of being re-formulated for final delivery as comprising a liquid, a suspension, a gel, a geltab, a semisolid, a tablet, a sachet, a lozenge, a capsule, or as an enteral formulation.

78. The composition of any one of claims 68-77, wherein the composition is formulated for multiple administrations.

79. The composition of any one of claims 68-78, wherein the composition further comprises a pharmaceutically acceptable excipient.

80. The composition of any one of claims 68-79, wherein the purified population of bacteria comprises bacteria from at least two genera or species, and wherein the ratio of the two bacteria is 1:1.

81. The composition of any one of claims 68-80, wherein the composition comprises at least 2 different species or genera of bacteria.

82. The composition of any one of claims 68-81, wherein the composition provides for an alpha diversity of at least 5 after administration to the subject.
